SEQUENCE LISTING (51 sequences)

SEQ ID NO: 1 - protein, 243 aa, Arabidopsis thaliana
MPEARDRTERPVDYSTIFANRRRHGILLDEPDSRLSLIESPVNPDIGSIGGTGGLVRGNFTTWRPGNGRGGHTPFRLPQGRENMPIVTARRGRGGGLLPSWYPRTPLRDITHIVRAIERRRGAGTGGDDGRVIEIPTHRQVGVLESPVPLSGEHKCSMVTPGPSVGFKRSCPPSTAKVQKMLLDITKEIAEEEAGFITPEKKLLNSIDKVEKIVMAEIQKLKSTPQAKREEREKRVRTLMTMR

SEQ ID NO: 2 - protein, 234 aa, Oryza sativa
MPEVRNSGGRAALADPSGGGFFIRRTTSPPGAVAVKPLARRALPPTSNKENVPPSWAVTVRATPKRRSPLPEWYPRSPLRDITSVVKAVERKSRLGNAAVRQQIQLSEDSSRSVDPATPVQKEEGVPQSTPTPPTQKALDAAAPCPGSTQAVASTSTAYLAEGKPKASSSSPSDCSFQTPSRPNDPALADLMEKELSSSIEQIEKMVRKNLKRAPKAAQPSKVTIQKRTLLSMR

SEQ ID NO: 3 - protein, 362 aa, Arabidopsis thaliana
MEGKFAISESTNLLQRIKDFTQSVVVDLAEGRSPKISINQFRNYCMNPEADCLCSSDKPKGQEIFTLKKEPQTYRIDMLLRVLLIVQQLLQENRHASKRDIYYMHPSAFKAQSIVDRAIGDICILFQCSRYNLNVVSVGNGLVMGWLKFREAGRKFDCLNSLNTAYPVPVLVEEVEDIVSLAEYILVVEKETVFQRLANDMFCKTNRCIVITGRGYPDVSTRRFLRLLMEKLHLPVHCLVDCDPYGFEILATYRFGSMQMAYDIESLRAPDMKWLGAFPSDSEVYSVPKQCLLPLTEEDKKRTEAMLLRCYLKREMPQWRLELETMLKRGVKFEIEALSVHSLSFLSEVYIPSKIRREVSSP

SEQ ID NO: 4 - protein, 383 aa, Arabidopsis thaliana
MEESSGLSSMKFFSDQHLSYADILLPHEARARIEVSVLNLLRILNSPDPAISDLSLINRKRSNSCINKGILTDVSYIFLSTSFTKSSLTNAKTAKAFVRVWKVMEICFQILLQEKRVTQRELFYKLLCDSPDYFSSQIEVNRSVQDVVALLRCSRYSLGIMASSRGLVAGRLFLQEPGKEAVDCSACGSSGFAITGDLNLLDNTIMRTDARYIIIVEKHAIFHRLVEDRVFNHIPCVFITAKGYPDIATRFFLHRMSTTFPDLPILVLVDWNPAGLAILCTFKFGSIGMGLEAYRYACNVKWIGLRGDDLNLIPEESLVPLKPKDSQIAKSLLSSKILQENYIEELSLMVQTGKRAEIEALYCHGYNYLGKYIATKIVQGKYI

SEQ ID NO: 5 - protein, 1330 aa, Arabidopsis thaliana
MFFQHSQLQNSDHLLHESMADSNHQSLSPPCANGHRSTISLRDDQGGTFCLICFSNLVSDPRIPTVHVSYALHQLSIAISEPIFLRTLLSSHIHFLVSPLVHALSSIDDAPIAIQIMDMISLLCSVEESSIGEDFVERISDQLSSGALGWSRRQLHMLHCFGVLMSCENIDINSHIRDKEALVCQLVEGLQLPSEEIRGEILFALYKFSALQFTEQNVDGIEVLSLLCPKLLCLSLEALAKTQRDDVRLNCVALLTILAQQGLLANSHSNSASSMSLDEVDDDPMQTAENVAARPCLNVLFAEAIKGPLLSTDSEVQIKTLDLIFHYISQESTPSKQIQVMVEENVADYIFEILRLSECKDQVVNSCLRVLDLFSLAEHSFRKRLVIGFPSVIRVLHYVGEVPCHPFQIQTLKLISSCISDFPGIASSSQVQEIALVLKKMLERYYSQEMGLFPDAFAIICSVFVSLMKTPSFGETADVLTSLQESLRHSILASLSLPEKDSTQILHAVYLLNEIYVYCTASTSINMTSCIELRHCVIDVCTSHLLPWFLSDVNEVNEEATLGIMETFHSILLQNSDIQAKEFAELLVSADWFSFSFGCLGNFCTDNMKQRIYLMLSSLVDILLEQKTGSHIRDALHCLPSDPQDLLFLLGQASSNNQELASCQSAALLIFHTSSIYNDRLADDKLVLASLEQYIILNKTSLICAISDSPALLNLVNLYGLCRSLQNERYQISYSLEAERIIFHLLNEYEWDLGSINIHLESLKWLFQQESISKSLIYQIQKISRNNLIGNEVHNVYGDGRQRSLTYWFAKLISEGDNYAATLLVNLLTQLAEKEEQENDVTSILNLMNTIVSIFPTASNNLSMNGIGSVVHRLVSGFSNSSLGTSFKTLLLLVFNILTSVQPAVLMIDESWYAVSIKLLNFLSLRDTAIKQNHEDMVVIGILSLVLYHSSDGALVEASRNIVSNSYLVSAINTVVDVACSKGPALTQCQDETNIGEALAFTLLLYFFSLRSLQIVLAGAVDWQAFFGTSTSLETLPVVCIYCHNLCRLMHFGAPQIKLIASYCLLELLTGLSEQVDIKKEQLQCSSSYLKSMKAVLGGLVFCDDIRVATNSALCLSMILGWEDMEGRTEMLKTSSWYRFIAEEMSVSLALPCSASSTYVNHHKPAVYLTVAMLRLKNKPVWLRTVFDESCISSMIQNLNGINISREIVILFRELMQAELLNSQQVTKLDRAFQECRKQMHRNGTRDETVEEQVQRKIPSIHDHSEFCNYLVHLMVSNSFGHPSESETYTQKKKQILDEMEQFSELISTREGRVSPIQEETRQMQTERIV

SEQ ID NO: 6 - protein, 378 aa, Arabidopsis thaliana
MSSSVAEANHTEKEESLRLAIAVSLLRSKFHNHQSSSSTSRCYVSSESDALRWKQKAKERKKEIIRLQEDLKDAESSFHRDLFPANASCKCYFFDNLGVFSGRRIGEASESRFNDVLRRRFLRLARRRSRRKLTRSSQRLQPSEPDYEEEAEHLRISIDFLLELSEADSNDSNFSNWSHQAVDFIFASLKKLISMGRNLESVEESISFMITQLITRMCTPFKGNEVKQLETSVGFYVQHLIRKLGSEPFIGQRAIFAISQRISILAENLLFMDPFDESFPEMDECMFILIQLIEFLICDYLLPWAENEAFDNVMFEEWIASVVHARKAVKALEERNGLYLLYMDRVTGELAKRVGQITSFREVEPAILDKILAYQEIE

SEQ ID NO: 7 - protein, 449 aa, Arabidopsis thaliana
MKMNINKACDLKSISVFPPNLRRSAEPQASQQLRSQQSQQSFSQGPSSSQRGCGGFSQMTQSSIDELLINDQRFSSQERDLSLKKVSSCLPPINHKREDSQLVASRSSSGLSRRWSSASIGESKSQISEELEQRFGMMETSLSRFGMMLDSIQSDIMQANRGTKEVFLETERIQQKLTLQDTSLQQLRKEQADSKASLDGGVKFILEEFSKDPNQEKLQKILQMLTTIPEQVETALQKIQREICHTFTREIQVLASLRTPEPRVRVPTAPQVKAKENLPEQRGQAAKVLTSLKMPEPRVQVPAAPQAKENFPEQRGPVAKSNSFCNTTLKTKQPQFPRNPNDASARAVKPYLSPKIQVGCWKTVKPEKSNFKKRATRKPVKSESTRTQFEQCSVVIDSDEEDIDGGFSCLINENTRGTNFEWDAEKETERILRTARRTKRKFGNPIIIN

SEQ ID NO: 8 - protein, 617 aa, Arabidopsis thaliana
MFYSHQLLARKAPLGQIWMAATLHAKINRKKLDKLDIIQICEEILNPSVPMALRLSGILMGGVVIVYERKVKLLFDDVNRFLVEINGAWRTKSVPDPTLLPKGKTHARKEAVTLPENEEADFGDFEQTRNVPKFGNYMDFQQTFISMRLDESHVNNNPEPEDLGQQFHQADAENITLFEYHGSFQTNNETYDRFERFDIEGDDETQMNSNPREGAEIPTTLIPSPPRHHDIPEGVNPTSPQRQEQQENRRDGFAEQMEEQNIPDKEEHDRPQPAKKRARKTATSAMDYEQTIIAGHVYQSWLQDTSDILCRGEKRKVRGTIRPDMESFKRANMPPTQLFEKDSSYPPQLYQLWSKNTQVLQTSSSESRHPDLRAEQSPGFVQERMHNHHQTDHHERSDTSSQNLDSPAEILRTVRTGKGASVESMMAGSRASPETINRQAADINVTPFYSGDDVRSMPSTPSARGAASINNIEISSKSRMPNRKRPNSSPRRGLEPVAEERPWEHREYEFEFSMLPEKRFTADKEILFETASTQTQKPVCNQSDEMITDSIKSHLKTHFETPGAPQVESLNKLAVGMDRNAAAKLFFQSCVLATRGVIKVNQAEPYGDILIARGPNM

SEQ ID NO: 9 - protein, 442 aa, Arabidopsis thaliana
MSSSSRNLSQENPIPRPNLAKTRTSLRDVGNRRAPLGDITNQKNGSRNPSPSSTLVNCSNKIGQSKKAPKPALSRNWNLGILDSGLPPKPNAKSNIIVPYEDTELLQSDDSLLCSSPALSLDASPTQSDPSISTHDSLTNHVVDYMVESTTDDGNDDDDDEIVNIDSDLMDPQLCASFACDIYEHLRVSEVNKRPALDYMERTQSSINASMRSILIDWLVEVAEEYRLSPETLYLAVNYVDRYLTGNAINKQNLQLLGVTCMMIAAKYEEVCVPQVEDFCYITDNTYLRNELLEMESSVLNYLKFELTTPTAKCFLRRFLRAAQGRKEVPSLLSECLACYLTELSLLDYAMLRYAPSLVAASAVFLAQYTLHPSRKPWNATLEHYTSYRAKHMEACVKNLLQLCNEKLSSDVVAIRKKYSQHKYKFAAKKLCPTSLPQELFL

SEQ ID NO: 10 - protein, 477 aa, Oryza sativa
MAAKRPAAGEGGGKAAAGAAAAKKRVALVNITNVAAAANNAKFNSATWAAPVKKGSLASGRNVCTNRVSAVKSASAKPAPAISRHESAPQKESVIPPKVLSIVPTAAPAPVTVPCSSFVSPMHSGDSVSVDETMSMCDSMKSPDFEYIDNGDSSSVLGSLQRRANENLRISEDRDVEETKWNKDAPSPMEIDQICDVDNNYEDPQLCATLASDIYMHLREAETRKRPSTDFMETIQKDVNPSMRAILIDWLVEVAEEYRLVPDTLYLTVNYIDRYLSGNEINRQRLQLLGVACMLIAAKYEEICAPQVEEFCYITDNTYFRDEVLEMEASVLNYLKFEVTAPTAKCFLRRFVRVAQVSDEDPALHLEFLANYVAELSLLEYNLLSYPPSLVAASAIFLAKFILQPTKHPWNSTLAHYTQYKSSELSDCVKALHRLFSVGPGSNLPAIREKYTQHKKFVAKKHCPPSVPSEFFRDATC

SEQ ID NO: 11 - DNA, 27 bp, Artificial Sequence
  Synthetic construct Primer 1
ggtgcgatttctccagcagtaaaaatc

SEQ ID NO: 12 - DNA, 26 bp, Artificial Sequence
  Synthetic construct Primer 2
ctgagaagatgaagcaccggcgatat

SEQ ID NO: 13 - DNA, 27 bp, Artificial Sequence
  Synthetic construct Primer 3
cacatactcgctactggtcagagaatc

SEQ ID NO: 14 - DNA, 22 bp, Artificial Sequence
  Synthetic construct Primer 4
ctgaagctgaaccttcgtctcg

SEQ ID NO: 15 - DNA, 20 bp, Artificial Sequence
  Synthetic construct Primer 5
aatccagatcccccgaatta

SEQ ID NO: 16 - DNA, 18 bp, Artificial Sequence
  Synthetic construct Primer 6
cagcagaacacccccatc

SEQ ID NO: 17 - DNA, 26 bp, Artificial Sequence
  Synthetic construct Primer 7
ctgagaagatgaagcaccggcgatat

SEQ ID NO: 18 - protein, 385 aa, Arabidopsis thaliana
MSSSVAEANHTEKEESLRLAIAVSLLRSKFQNHQSSSSTSRCYVSSESDALRWKQKAKERKKEIIRLQEDLKDAESSFHRDLFPANASCKCYFFDNLGVFSGRRIGEASESRFNDVLRRRFLRLACVVILSLARRRSRRKLTRSSQRLQPSEPDYEEEAEHLRISIDFLLELSEADSNDSNFSNWSHQAVDFIFASLKKLISMGRNLESVEESISFMITQLITRMCTPVKGNEVKQLETSVGFYVQHLIRKLGSEPFIGQRAIFAISQRISILAENLLFMDPFDESFPEMDECMFILIQLIEFLICDYLLPWANEAFDNVMFEEWIASVVHARKAVKALEERNGLYLLYMDRVTGELAKRVGQITSFREVEPAILDKILAYQEIE

SEQ ID NO: 19 - protein, 383 aa, Populus trichocarpa
MASSEPATDTKTASSPTDDQSLKLAVAISLLRSKLLQKQPPPPPPPSNPPSESDALRWKRKAKERKQELLRLREDLREAEDASQCDLFPQTALCKCYFFDNLGKSSPKPVGDGSDRRFNDILRRRFLRQVRIKERRKRINNSNIKIRFSDIYSKNEAEQLRAAVDFLVELCDTTSPGRVEEANFANWSHQAADFILASLRNLLSIGNNMELIEGIVSRLIVRLVKRMCSPSHGDESRQTDTDTQFYIQQLIRKLGCEPHIGQRAILSVSQRISMVAENLLFLDPFDEAFSNMHECLFIMIQLIEFLISDYLLTWSRDEGFDHVLFEEWVTSVLHARKALELLESRNGLYVLYMDRVTGELAKHVGQVSSFQKLSQDILDNLFC

SEQ ID NO: 20 - protein, 363 aa, Vitis vinifera
MSTSNTDSHQSLKLAVAMALLRSKLLHNTNPPPPHSDALRWKRKAKERKQELLRLKEDLREAEDGLRHDLFPPSASCKCHFFDDLGKLSPNQFERGSNRNFNDVLRRRFLRQVRLKERRRKRTDDSIKHNHYSDIVCEDETEQLRASIDFLVELCDTASPNSNFTNWSHQAVDFILASLKNLLSVRKNVEYIKGIINSLIKHLVRRLCTPLKGDELHHLDADHQFYVQHLIRKLGSDPFVGHRAILSVSQRISLIAESLLFLDPFDDAFPNLHGCMFVLIQLIEFLISDYFLVWSRDEGFDNMLFVEWVTSILHARKALELLESRNGLYVLYMDRVTGELAKHVGQVSLLQELNPDIINILFH

SEQ ID NO: 21 - protein, 423 aa, Oryza sativa
MAPPASRPPTPTPTPTANAAASSSRIESPSLRAALAMALIHYNRLPSRAAAAAAPSPQALLNWKRKAKDRKREILRLREELKLLQDGARGEEMEPPVASCRCHFFDGCGDLPPPTDGDAGEHWVDDVLRRRFVRLEYNTEDEVQQLSLSIDFLVELSDGLFAKREAGSSFTTFSHQAVDFILASLKNILSSEREKEIIEEIINGLVARLMKRMCTTPENAGSVDCSDAQFSLQHLFRKLGNEEFVGQRIILAISQKISNVSEKLLLADPFDDGFPEMHSNMFIMIQLIEFLISDSFNNWLCRDHFDRKLFEEWVRSILKARKDLEVLDGRNGLYVVYIERVIGRLAREVAPAAHQGKLDLEDGSTMWSMRYLRPHEAIELATSTDSPCILVIGGCLPLFVSPTKKEKKEALDSTARCFASLLA

SEQ ID NO: 22 - protein, 391 aa, Zea mays
MALPKPRPPTPTASAATGTSSSRIDSPSLKAALAMALIHYNRLPGKANATAGTSPPSLLHWKRKAKDRKREILRLREELKVLQDGVRGEEMEPPVASCRCHFFDGCRDLRPQQGGGGGEHWVDEVLRRRFLRLVRWKEKRRRVDRSLPSSSLIDFNSEDEMQQLSMSTDFLVELSDGIFAKSEAGHSFATFSHQAVDFILATLKNILSSEREKDLVGEIIDSLVTRLMKRMCTVPEKLVTSDSGSTGCSDAQFSVQHLFRKLGNDEFFGQRVILVVSQKISNVSERLFLADPFADAFPDMHDNIFIMIQLLEFLISDYMKVWLCCEHINKRLFEECTRSILKARNDLQILENMNGLYVVYIERVVGRLARDVAPAAHQGKLDLEVFSKLLC

SEQ ID NO: 23 - protein, 443 aa, Arabidopsis lyrata
MSSSSSSKNLSQENPIPRPNLAKTRTSLRDVGNRRVPLGDITNQKTGSRNSSSSSTLVHCSNKISQSKKASKPALSRNWNLGILDCGLPPKSNANSNIIVPYEDTELPQIDDSLLSSSPGLSVDASPTHSDPSISTHDSLKSHIVEHMVESSTDDGNDDDEIVNIDSDLMDPQLCASFAFDIYEHLRASEVKKRPALDYMERIQLNINASMRSILIDWLVEVAEEYRLSPETLYLAVNYVDRYLTGNAINKQNLQLLGVACMMIAAKYEEVCVPQVEDFCYITDNTYLRNELLEMESSVLNYLKFELTTPTAKCFLRRFLRAAQGRKEVPSLLSECLACYLTELSLLDYAMLRYAPSLVAASAVFLAQYILHPSRKPWNATLEHYTSYRAKHMEACVKNLLQLCNEKPSSDVVAIRKKYSQHKYKFAAKKLCPTSLPQELFLC

SEQ ID NO: 24 - protein, 249 aa, Arabidopsis lyrata
MPEARDRIERPVDYPAIFVNRRSNGVLLDEPDSRLSLIESPVNPETGSMGRGSLVGTGGLVRGNFSTWRPGNGRGGHSPFRLSQGRENNMPMVSARRGRGPSLLPSWYPRTPLRDITHIMRTIERRRGAGIGGDDGRDIEIPTHQQVGVLESPVPLSGEHKCSIVTPGPSVGFKRSCPPSTAKVHKMLLDITKEIAEEEAGFITPEKKLLNSIDKVEKIVMAEIQKLKSTPHAKREEREKRVRTLMSMR

SEQ ID NO: 25 - protein, 249 aa, Brassica rapa
MAEARDRLEKPVDYAAIFANRRSHGVLLDEPEAGLGVLEHPVRRLPSGSRVYPQPGGNYSSWRPGHGNGSGQSPFRFSQGRENVTMASARRGRGGASGSLLPSWYPRTPLRDITHIMRAIERKRRAGMGVESALGGETPSHQQVRFLETPVALAEDEHNCVMVTPAPAVGLKRSCPPSTAKVHKMLLDITKDISDNDEQARFITPEKKLLNSIDVVEKIVMAEIQKLKSTPLAKRQEREKRVKTLMSMR

SEQ ID NO: 26 - protein, 259 aa, Arabidopsis thaliana
MPEARDRIERQVDYPAAFLNRRSHGILLDEPATQHNLFGSPVQRVPSEATGGLGSIGQGSMTGRGGLVRGNFGIRRTGGGRRGQIQFRSPQGRENMSLGVTRRGRARASNSVLPSWYPRTPLRDISAVVRAIERRRARMGEGVGRDIETPTPQQLGVLDSLVPLSGAHLEHDYSMVTPGPSIGFKRPWPPSTAKVHQILLDITRENTGEEDALTPEKKLLNSIDKVEKVVMEEIQKMKSTPSAKRAEREKRVRTLMSMR

SEQ ID NO: 27 - protein, 258 aa, Arabidopsis lyrata
MPEARDRIERPVDYPAAFLNRRSHGILLDEPATHHNLFGSPVQRVPSEATGLGSVGQGSMMGRGGLVRGNFGIRRTGGGRRGQIQFRSPQGRENMSLGVTRRGRARASNSVLPSWYPRTPLRDVSAVVRAVERRRARMGEGVGRDIETPTPQQLGVLDSLVPLSGAQLEHDYSMVTPGPSVGFKRPWPPSTAKVHQILLDITRENTGEEDALTPQKKLLNSIDKVEKVVMEEIQKMKSTPSAKRAEREKRVRTLMSMR

SEQ ID NO: 28 - protein, 223 aa, Brassica rapa
MPEARDRRERSVDYPAAFLNRRSHGILLDESPLRSPVQRLPSSESLVFGRGGFARGNLGIRRTGGGGGRRRGRARASASVLPSWYPRTPLRDVSSVVRAIERRRARVGDVETPTPQQLEVVLDDSLAPVSGERNYSMVTPGPSVGFKRPWPPSTAKVHQILLDITRQSSAEEEEEALTPQKKLLNSIDKVEKVVMEEIQKMKSTPSAKRAEREKRVRTLMSMR

SEQ ID NO: 29 - protein, 247 aa, Populus trichocarpa
MTESRDRLSRAVDIAAIFAARRQSMNLGIYQDRPELDMALFGSPRTNTAIRNQTVGVGTITGRGRGRLGTPRGRGGWTPLDRENMPPPGSARRRRGRGSNSLLPSWYPRTPLRDITAVVRAIERRGRLGGSDGREIGSPMPQGRMDPEFSEATPVAHPEPSNRIMSPKPTPAFKGCPSTIGKVPKILQHITNQASGDPECLTPQKKLLNSIDTVEKVVMEELQKLKRTPSAKKAEREKRVRTLMSMR

SEQ ID NO: 30 - protein, 248 aa, Populus trichocarpa
MPVSRDRLSSPVDIAALFAARRQSRILGVYQDQPELDMALFGSPRPNAATRTQTVGAGTIAVRGRGGLGTPRGRGGRTTLGRENIPPPGSARRGRGRGSNSVLPAWYPRTPLRDVTAVVRAIERRRERLGGSDGLEIRSPMPQVRMNHDSSEATPVAHLEHSNRIMSPKPTTAVKGCSSTIGKVPKILQHITNQASGDPDSLTPQKKLLNSIDTVEKVVMEELRKMKRTPSARKAEREKRVRTLMSMR

SEQ ID NO: 31 - protein, 266 aa, Vitis vinifera
MPESRDRLSRPEDIAELFLRRRSGILGILADGSERSSNLFASPSRRETTTRTTTLGARGATGILASRGGGVGRGGFGTPRIGTGRGRGRAVYRSPLFGRENTPATGSGRRGRGRSGNSVLPSWYPRTPLRDITHVVRAIERRRARLREIDGQQIDIPIPQDISDVHDPILPPSSAQLEQDISMISPSPTSGMKLVPKAVGKVPKILLDITDQTGGGSDFLTPQKKLLNSIDTVEKAVMDELGKLKRTPSAKRAEQEKRVRTLMSMR

SEQ ID NO: 32 - protein, 210 aa, Glycine max
MPQSRHRRVTVVDLAASLARRRVSFIFNEAPTLRTPPRTAAFGRGRARASPRSQNIPPSTARRGRGRVPLRSVLPAWFPRTPLRDITAVVQAIERRSARLGEVEGQRIGNTDPASDRLVSEPSEPASASASASAVKSPKSVGVKLRTPFGSKVPKIFLDISELPEHDESEALTPQKKLLDNIDQVEEAVREELNKLKRTPSAKKTEREKR

SEQ ID NO: 33 - protein, 215 aa, Glycine max
MPESRDRRITVVDLAAAIARRRASFIYIDSPPLRTPQRTAAIGRGRASGSPGSQNTPPSTARRGRGRVPSRNVLPAWYPRTPLRDITVVVQAIERRRARSGEAEGQRIGSTDPASDRLVTEPSEPASADSAVKSPKSVGVKLRTPFGSKVPKIFLDISELPEDDESETLTPQKKLLNNIDQVEEAVREELKKLKRTPSAKKAEREKRVRTLMSMR

SEQ ID NO: 34 - protein, 216 aa, Oryza sativa
MPEMRDSKRTALGELSGGGGFFIRRVASPGALAARGPGKPLARRFIRPSNNKENVPPVWAVKATATKRRSPLPDWYPRTPLRDITAIAKAIQRSRLRIAAAQQRSQTPEQNTPHCTEVRDSLDVEPGINSTQIVATPASSLAKDSLKIFSSPSETSLVTPSKPMDPVLLDDMEKKLSSSIEQIEKMVRRNLKRTPKAAAAQPSKRAIQRRTLMSMR

SEQ ID NO: 35 - protein, 231 aa, Sorghum
MPDSRDGRRAALADLSSGVGGGGFFIRRVASPRALAVRGAGKPLARRYMSPSRNKENLLPIWALRATPAKRSPLPGWYPRTPLRDITAIAKAIQRSRARIAAAQQQSQRIEQSPQSVNVTTPAQAEQDAPHIAEASHAVASGSGSTERETVANPATVLADDNLNVSSSPAESSLNTPSKPMDPALADIVEKKLSSSIEKIEKLVRKNMKRTPKAARASRRATQRRNLMSMR

SEQ ID NO: 36 - protein, 225 aa, Sorghum
MPQLRTASRPVLARNSTGGIFIRRRVASPGGAVKPLARRVRTHFSNKENVPPVGAARAKPKRRSPLPDWYPRSPLRDITSIVKALEKRNRLEEDAARQHIQWNEDSPQPVDPTTTVHAEHSDPDSQSTQTQETLGVVASPGSTSAVANNVTSVAEDKQEASSSPSDCLQMAPSKPNDPSPADLEKKMSSSIEQIEKMVRRHMKETPKAAQPSKLVVQRRILMSMR

SEQ ID NO: 37 - protein, 216 aa, Sorghum
MHESRTARRPALADISGGGFFIRRVESPGAVLVKGAVKPLARRALSQSSNKENVPPVGAVRGAPKRKSPLPDWYPRTPLRDITSIVKAIERRSRLQNAATEQTILWTEDSSQSVDPITPASAEQGVPTIEGGQAVARHATSLGDGKLKTSSSPFDCSLQATPSKPNDPALADLMEKKLSNSIEQIEKMVRRNLKKTPKAAQPSKRTIQSRILMSMR

SEQ ID NO: 38 - protein, 227 aa, Zea mays
MPESRDGRSEDLADLSGGVGGGGFFIRRVASPGALAVRGVRKPLARRYISPSRNKENLLPVWALRVTPTKRSPLPGWYPRTPLRDITAIAKAIQRSRSRIAAAQQRSQRIEQSSQSVNVTTPAQAEQDAHIAEASHAVASGSGSTEREAVANPATVLADDNLNVSSLAAEGSLNTPSKPMDPALADKKLSGSIEKVEKLVRKNLKRTSRAAQASRRATQRRNLMSMR

SEQ ID NO: 39 - protein, 215 aa, Zea mays
MPQLRTASRPALASNSAGGFFIRRRVASPGTSQAKGAAKPLARRVRTPAARAKPKRRSPLPDWYPRVPLRDITSIVKALEKRNRLEEDAARQHIQSNEDSSQPVDPTTAEHSDPDSQSTQTQETPGAVASGPSSTSAVANRVTSVAEGKQEATDCSLQVAPSKPNDPSPADLEKKLSGSIEQIEKMVRRHMKETHPKAAQPSKVVVQRRILMSMR

SEQ ID NO: 40 - protein, 195 aa, Zea mays
MLEVRTARRPALADISGGGFFMRTVESPGAVLVNGAVKRPARQFLSPSSNKENVPPVGAFRATPKRRTPLPDWYPRTPLRDITSIVKAIERRRSRLQNAAAQQQIQWTEDPSRSVDPITPVQAEQGGVPTTVDGQGVGSPATCLEDGKLKTSSYPSSDCSLQATPSKPNDPALADLVEKRLSSSIEQIEKMVRRT

SEQ ID NO: 41 - protein, 238 aa, Medicago truncatula
MPEARDRRVIPLDVDTLFRRPFSAVFQESEPLSVTPAPAPFTAGLDLFFTERTPVRREVARARRSPGSENTPPTTARRGRGRATASRSALPSWYPRTPLQDITAIVRAIERRRERQGTEEIEQTGTPVHANQLTIFSDPSSFSAAIGSSSRVHKKSPKSCIKLKTPYGSKVPKIIIDIAKLPAAEDGESELLTPQKKLLHSIDIIEREVKQELMKLKRTPTAKKAEHQKRVRTLMSMR

SEQ ID NO: 42 - protein, 238 aa, Mallus
MPEARDRLSRPVDLATAYAQRLAGNRRVYIDLPEQTILAFSPPVRLPTGLGIGATGVVGVGGLPRSSLRTPRTVTGRGRISFRLSTVDRENTPSGSSHRRRGRSSNSVLPSWYPRTPLHDITAVTRAIERRRARLAESNGENTEGQAPQDQNALDQSLPVLGAQFDHGVPVTPYSALRTKRRLPPPVVKVQKIIRDVSNQPSEGEFLTPQKKLMNSIDMVEEVVRKELDRLKRTPSAK

SEQ ID NO: 43 - protein, 193 aa, Mallus
GRLPRSILRTPRTVTGRGRIPFRLSTVDRENTPRGSSHQRGGRASNSVLPYWYPRSPLQDITAVVRAIESRRARLIESDGQNTEGQVPQDQNALDQSLPVSGAQFDHGVPMTPYSAVRTKHCLPPSVGKVQQILRDVSNQPSEGEFLTPQKKLMNSIDMVEKVVTKELERLKRTPSSKKAEREQKVRTLMSMR

SEQ ID NO: 44 - protein, 250 aa, Ricinus communis
MPEARDRLSRPIDIATVFSRRRSGLIGVYQDQPDLETALFGSPITSRLDTATRTGTVGLSPRGRGRGSFGTPRNQTLRGRHPYVTIGRENTPVTGRRGNGNRSVLPSWYPRTPLRDITAIVRAIERRRELLGEGRAQEIESPVPHAYEVPDSSEPSAVAHLEHSNSMMSPIPSLQVKRCPPTVGKVSKILLDITNKASDDSEFLTPQKKLLNSIDTVEKEVMEELRKLKRTASAKKAEREKKVRTLMSLR

SEQ ID NO: 45 - protein, 250 aa, Lycopersicon esculentum; identical to SEQ ID NO: 44

SEQ ID NO: 46 - protein, 249 aa, Melon
MSEARDRLERQVDYAEVFARRRSEGILDEQEMGSNLIGTPIARATTTTAAQQRPTNPGPGGGGANLRRTFGSPISGGIGRNRFLYRTPVLSRENPSAGSSRRSRSRGRNSVLPIWYPRTPLRDITAVVRAIERTRARLRENEGQGSDSSPAPSDERALEYSVSVASDHQEPIISLLTPKPTVGKVPKILRGIANENTVGAETLTPQKKLLNSIDKVEKVVMEELQKLKRTPSAKKAEREKRVRTLMSFR

SEQ ID NO: 47 - protein, 1176 aa, Oryza sativa
MSVQLHCLGILLNSTKDAATYIGDKQSLYLNLVNNLRLPRLIPLHIDTFLALRITLSDSIINLFWYSDEIRGEILFVLYKLSLLNATPWDDICDNDNVDLSAIGRSLLQFSLEVLLKTQNDDVRLNCIALLLTLAKKGAFDILLLSDPSLINSAEAEDNVPLNDSLVILFAEAVKGSLLSTNIEVQTGTLELIFHFLSSDANIFVLKTLIDQNVADYVFEVLRLSGNNDPLVISSIKVLSILANSEERFKEKLAIAVSTLLPVLHYVSEIPFHPVQSQVLRLVCISIINCSGILSLSQEEQIACTLSAILRRHGNGELGMSSETFALVCSMLVEILKLPSADDIQKLPSFIVEASKHAISLTFSHEYDCLFLIPHSLLLLKEALIFCLEGNKDQILRKKSLEDSIIETCETYLLPWLESAIVDGNDEETLSGILQIFQIILSRASDNKSFKFAEMLASSSWFSLSFGFMGLFPTDHVKSAVYLVISSIVDKVLGISYGETIRDACIYLPPDPAELLYLLGQCSSEDFNLASCQCAILVILYVCSFYNERLAADNQILASVEQYILLNGAKFPHEIPGSLMLTLLVHLYAFVRGISFRFGIPHSPEAEKTLFHAMTHKEWDLLLIRVHLIALKWLFQNEELMEPLSFHLLNFCKFFCEDRTVMLSSSTQLVDIQLIAELVYSGETCISSLLVSLLSQMIKESAEDEVLSVVNVITEILVSFPCTSDQFVSCGIVDALGSIYLSLCSSRIKSVCSLLIFNILHSASAMTFTCDDDAWLALTMKLLDCFNSSLAYTSSEQEWKILIGILCLILNHSANKVLIEPAKAIILNNCLALLMDGIVQEACAKGPSLFQHNQETTFGELLILMLLLIFFSVRSLQAILEASIDWQEFLQYSDDTESSSVLGIPCHDLCRLMHFGPSPVKLIASQCLLELLNRISDQRSCLNAELRCSAKYLKSMIAVTEGMVFDQDSRVAENCGACLTVILGWERFGSREKAVIRESKWSRLILEEFAVALTAPGLTSKSFSNQQKIAANIALSLLQLSQVPDWLTSLFSDSLISGIVANLSARNVTAEIVTLFSELMAKNYLNQEHIAGLHNLFQVCRRQAYEGGGGSKAQPSEQKAAAARCADDVRALLFGMMLEQRACSRATVEMEQQRLLREIDSFFFQESSLREQNSVK

SEQ ID NO: 48 - protein, 381 aa, Oryza sativa
MAGREKRRRVAALDGEERRRRQEEAATLLHRIRGLVRWVVAEVAAGRSPTVALHRYQNYCSSASAAAASPCACSYDVPVGTDVLSLLHRGSHASRLNVLLRVLLVVQQLLQQNKHCSKRDIYYMYPSIFQEQAVVDRAINDICVLFKCSRHNLNVVPVAKGLVMGWIRFLEGEKEVYCVTNVNAAFSIPVSIEAIKDVVSVADYILIVEKETVFQRLANDKFCERNRCIVITGRGYPDIPTRRFLRYLVEQLHLPVYCLVDADPYGFDILATYKFGSLQLAYDANFLRVPDIRWLGVFTSDFEDYRLPDCCLLHLSSEDRRKAEGILSRCYLHREAPQWRLELEAMLQKGVKFEIEALSACSISFLSEEYIPKKIKQGRHI

SEQ ID NO: 49 - protein, 385 aa, Oryza sativa
MAEAGVAAASLFGADRRLCSADILPPAEVRARIEVAVLNFLAALTDPAAPAISALPLISRGAANRGLRRALLRDDVSSVYLSYASCKRSLTRANDAKAFVRVWKVMEMCYKILGEGKLVTLRELFYTLLSESPTYFTCQRHVNQTVQDVVSLLRCTRQSLGIMASSRGALIGRLVVQGPEEEHVDCSILGPSGHAITGDLNVLSKLIFSSDARYIIVVEKDAIFQRLAEDRIYSHLPCILITAKGYPDLATRFILHRLSQTYPNMPIFALVDWNPAGLAILCTYKYGSISMGLESYRYACNVKWLGLRGDDLQLIPQSAYQELKPRDLQIAKSLLSSKFLQDKHRAELTLMLETGKRAEIEALYSHGFDFLGKYVARKIVQGDYI

SEQ ID NO: 50 - protein, 492 aa, Oryza sativa
MKLKMNKACDIASISVLPPRRTGGSSGASASGSVAVAVASQPRSQPLSQSQQSFSQGASASLLHSQSQFSQVSLDDNLLTLLPSPTRDQRFGLHDDSSKRMSSLPASSASCAREESQLQLAKLPSNPVHRWNPSIADTRSGQVTNEDVERKFQHLASSVHKMGMVVDSVQSDVMQLNRAMKEASLDSGSIRQKIAVLESSLQQILKGQDDLKALFGSSTKHNPDQTSVLNSLGSKLNEISSTLATLQTQMQARQLQGDQTTVLNSNASKSNEISSTLATLQTQMQADIRQLRCDVFRVFTKEMEGVVRAIRSVNSRPAAMQMMADQSYQVPVSNGWTQINQTPVAAGRSPMNRAPVAAGRSRMNQLPETKVLSAHLVYPAKVTDLKPKVEQGKVKAAPQKPFASSYYRVAPKQEEVAIRKVNIQVPAKKAPVSIIIESDDDSEGRASCVILKTETGSKEWKVTKQGTEEGLEILRRARKRRRREMQSIVLAS

SEQ ID NO: 51 - protein, 608 aa, Oryza sativa
MFYSHQLLARKAPLGQIWMAATLHSKINRKRLDKLDIIKICEEILNPSVPMALRLSGILMGGVAIVYERKVKALYDDVSRFLIEINEAWRVKPVADPTVLPKGKTQAKYEAVTLPENIMDMDVEQPMLFSEADTTRFRGMRLEDLDDQYINVNLDDDDFSRAENHHQADAENITLADNFGSGLGETDVFNRFERFDITDDDATFNVTPDGHPQVPSNLVPSPPRQEDSPQQQENHHAASSPLHEEAQQGGASVKNEQEQQKMKGQQPAKSSKRKKRRKDDEVMMDNDQIMIPGNVYQTWLKDPSSLITKRHRINSKVNLIRSIKIRDLMDLPLVSLISSLEKSPLEFYYPKELMQLWKECTEVKSPKAPSSGGQQSSSPEQQQRNLPPQAFPTQPQVDNDREMGFHPVDFADDIEKLRGNTSGEYGRDYDAFHSDHSVTPGSPGLSRRSASSSGGSGRGFTQLDPEVQLPSGRSKRQHSSGKSFGNLDPVEEEFPFEQELRDFKMRRLSDVGPTPDLLEEIEPTQTPYEKKSNPIDQVTQSIHSYLKLHFDTPGASQSESLSQLAHGMTTAKAARLFYQACVLATHDFIKVNQLEPYGDILISRGPKM

(19) United States
(12) Patent Application Publication
(10) Pub. No.: US 2014/0298507 A1
(43) Pub. Date: Oct. 2, 2014

(54) Synthetic Clonal Reproduction Through Seeds

(71) Applicant: Mark D. Spiller, Davis, CA (US)

(72) Inventors: Simon R. Chan, Davis, CA (US); Ravi Maruthachalam, Davis, CA (US); Raphael Mercier, Fontenay-le-Fleury (FR); Fabien Nogue, Orgerus (FR)

(21) Appl. No.: 13/991,078

(22) PCT Filed: Nov. 30, 2011
(86) PCT No.: PCT/US11/62718

§ 371 Date: Apr. 28, 2014

Related U.S. Application Data
(60) Provisional application No. 61/418,792, filed on Dec. 1, 2010.

(51) Int. Cl. A01H 5/10

(52) U.S. Cl. CPC A01H 5/10
USPC 800/260; 800/275; 800/298; 435/410

(57) ABSTRACT

Clonal embryos or seeds produced by conversion of apomeiotic gametes into clonal embryos or seeds. Clonal embryos or seeds are produced by crossing a MiMe plant, as either a female or male, with an appropriate plant which induces genome elimination (genome eliminator, GE). MiMe plants are those in which meiosis is totally replaced by mitosis. In specific embodiments MiMe plants are MiMe-1 plants or MIME-2 plants. In specific embodiments MiMe plants are mutant plants. In a more specific embodiment, the genome eliminator is a haploid inducer exhibiting directed genome elimination of its own genome.

### CROSS-REFERENCE TO RELATED APPLICATIONS

This application claims the benefit of U.S. provisional application 61/418,792, filed Dec. 1, 2010. This application is incorporated by reference herein in its entirety.

### STATEMENT REGARDING GOVERNMENT FUNDING

This invention was made with government support under Grant No. 1026094 awarded by the National Science Foundation. The government has certain rights in the invention.

### BACKGROUND OF THE INVENTION

Sexual reproduction in flowering plants involves two fertilization events: fusion of a sperm cell with the egg cell to give a zygote; and fusion of a second sperm nucleus with the central cell nucleus which initiates development of endosperm, the embryo nourishing tissue. Apomixis in nature occurs by a range of alterations to the regular sexual developmental pathway (FIG. 1). The principal functional components of apomixis include (i) the formation of an unreduced female gamete that also retains the parental genotype (apomeiosis), (ii) embryo development without fertilization of the egg cell by sperm (parthenogenesis) and (iii) endosperm development with or without fertilization of the central cell (pseudogamous or autonomous apomixis, respectively) [Bicknell, R. A. & Koltunow, A. M. (2004)].

Apomixis, asexual reproduction through seeds, results in progeny that are genetic clones of the maternal parent [Bicknell, R. A. & Koltunow, A. M. (2004), Koltunow, A. M. & Grossniklaus, (2003)]. Cloning through seeds has potential revolutionary applications in agriculture because its introduction into sexual crops would allow perpetuation of any elite heterozygous genotype [Spillane, C. et al (2004), Spillane, C. et al. (2001)]. However, despite the natural occurrence of apomixis in hundreds of plant species, very few crop species reproduce via apomixis and attempts to introduce this trait by conventional breeding have failed [Spillane, C. et al. (2001), Savidan, Y. (2001)].

An alternative approach is to de novo engineer the production of clonal seeds [Spillane, C. et al (2004)]. A major component of apomixis, the initiation and formation of functional apomeiotic female gametes that are also genetically identical to the parent plant (apomeiosis), can be induced in a sexual plant using Arabidopsis thaliana mutants that affect meiosis (MiMe-1 or MiMe-2) [d'Erfurth, I. et al. (2009), or d'Erfurth, I. et al. (2010), respectively]. Apomeiotic gametes in these MiMe lines participate in sexual reproduction, giving rise to an increase in ploidy. In order to produce a clonal seed, apomeiotic female gametes must initiate embryo development without fertilization.

The controls governing the other steps of apomixis, initiation of egg cell and central cell division to begin seed development, are poorly understood. Mutations that mimic embryo development without fertilization (parthenogenesis) or those that initiate autonomous endosperm have been reported in Arabidopsis, but these genetic manipulations do not lead to the formation of viable seed [Guitton, A. E. & Berger, F. (2005), Rodrigues, J. C. et al. (2010)].

Here, the inventors demonstrate an alternative to seed development without fertilization, the conversion of apomeiotic gametes into clonal seeds by fertilizing them with a strain whose chromosomes are engineered to be eliminated from the resultant progeny. FIG. 2 schematically illustrates the formation of clonal seeds through a combination of formation of diploid gametes with genome elimination. In natural apomicts, unreduced clonal female gametes develop into embryos without fertilization. The alternative method of this invention to create clonal seed is to fertilize unreduced clonal gametes with gametes whose chromosomes are modified to be eliminated after fertilization. Directional genome elimination is induced by haploid inducers.

Directional genome elimination occurs in certain wide crosses (both interspecific and intergeneric), and leads to the formation of haploid plants [Dunwell, J. M. (2010), Bains, G. S. & Howard, H. W. (1950), Barclay, I. R. (1975), Burk, L. G. et al. (1979), Clausen, R. E. & Mann, M. C. (1924), Hougas, H. W. & Peloquin, S. J. (1957), Kasha, K. J. & Kao, K. N. (1970).]. The molecular basis for genome elimination is not understood, but one theory posits that centromeres from the two parent species interact unequally with the mitotic spindle, causing selective chromosome loss [Bennett, M. D., et al. (1976); Finch, R. A. (1983), Laurie, D. A. & Bennett, M. D. (1989)].

Haploid inducer plants which induce genome elimination have been reported, particularly in maize [U.S. Pat. Nos. 5,749,169 and 5,639,95; published International applications WO 2005/004586 and WO 2008/097791, Barret, P. et al. (2008); Röber, F. K. et al. (2005), Lashermes, P. & Beckert, M. (1988)]. Many haploid inducers exhibit low rates of haploid induction. It has recently been shown that haploid plants can be generated through seed by altering the centromeric-specific histone variant CENH3 in Arabidopsis. Mutants expressing certain altered CENH3 proteins when crossed to wild-type exhibit function as haploid inducers in which progeny preferential eliminate chromosomes originating from the cenh3 mutant parent [Ravi, M. & Chan, S. W. (2010), Ravi, M., et al. Jul. 13, 2010]. The genome elimination strain GFP-tailswap was reported as having a very high frequency of generation of haploid plants (25-45%) in crosses to wild-type as the pollen donor. However, GFP-tailswap plants were reported to be mostly male sterile making crosses with female mutants difficult. In addition, GFP-tailswap plants were reported to give an extremely low frequency of viable seeds when crossed as the female to a tetraploid male that produces diploid gametes.

### SUMMARY OF THE INVENTION

The present invention relates to the production of clonal embryos or seeds by conversion of apomeiotic gametes into clonal embryos or seeds. More specifically, clonal embryos or seeds are produced by crossing a MiMe plant, as either a female or male, with an appropriate plant which induces genome elimination (genome eliminator, GE). MiMe plants are those in which meiosis is totally replaced by mitosis. In specific embodiments MiMe plants are MiMe-1 plants. In specific embodiments MiMe plants are MiMe-2 plants. In specific embodiments MiMe plants are mutant plants. In a more specific embodiment, the genome eliminator is a haploid inducer exhibiting directed genome elimination of its own genome. More specifically, the genome eliminator exhibits a haploid production rate of 1% or higher viable haploids and more preferably exhibits 10% or higher viable haploids when crossed with its corresponding wild-type. In another specific embodiment, the genome eliminator is a plant that expresses one or more altered CENH3 proteins, for example GFP-tailswap or GFP-CENH3. In a specific embodiment, the genome eliminator is a mutant plant or progeny thereof. In a specific embodiment, the genome eliminator is a transformed plant or progeny thereof.

In one aspect, the present invention relates to use of efficient genome elimination strains having altered CENH3 proteins with improved fertility and seed viability (compared to GFP-tailswap) for production of clonal embryos or seeds. In specific embodiments, the genome eliminator is a plant that expresses one or more altered CENH3 proteins. In specific embodiments, the genome eliminator is a plant that expresses two or more altered CENH3 proteins. In specific embodiments, the genome eliminator is a plant that expresses two altered CENH3 proteins, one of which proteins is GFP-CENH3. In another specific embodiment, the genome eliminator is a plant that expresses two altered CENH3 proteins, one of which proteins is GFP-tailswap. In another specific embodiment, the genome eliminator is a plant that expresses at least two altered CENH3 proteins, one of which proteins is GFP-tailswap and another of which is GFP-CENH3.

The invention also relates to clonal progeny produced by crossing a MiMe plant with a genome eliminator plant and to plant cells and tissue of such progeny. In specific embodiments the progeny are produced by crossing a MiMe plant with a genome eliminator which is a plant that expresses one or more altered CENH3 proteins.

In specific embodiments, MiMe plants form asexual diploid gametophytes which are then pollinated with pollen of the genome eliminator, the chromosome of the genome eliminator is selectively eliminated and an embryo develops solely from the diploid egg cell genome (gynogenesis). In other specific embodiments, genome eliminator plants form haploid gametophytes which are double fertilized by diploid pollen of a MiMe plant, the maternal genome of the genome eliminator is selectively eliminated and a diploid embryo develops from the sperm cell (androgenesis).

In specific embodiments, the MiMe plants and genome eliminator plants are Arabidopsis, particularly Arabidopsis thaliana. In specific embodiments, the MiMe plants and Arabidopsis plants are Oryza sativa. In specific embodiments, the MiMe plants and genome eliminator plants are Zea mays.

The invention relates to a method for generating clonal embryos or clonal seed which comprises the steps of crossing a MiMe plant as a male or female with a genome eliminator plant and selecting viable clonal embryos or seeds.

The invention also relates to methods of cultivating a clonal plant that is obtained by the methods of this invention and recovering gametes, particularly viable gametes, produced by that plant.

Plants produced by the methods of this invention are for example useful in plant breeding.

Other aspects of the invention will be apparent to one of ordinary skill in the art on consideration of the following detailed description, examples and figures. It is to be understood, however, that this detailed description, as well as any examples and figures are exemplary only and do not limit the invention.

### BRIEF DESCRIPTION OF THE DRAWINGS

FIG. 1 illustrates an overview of sexual, and asexual development and provides a comparison to an exemplary synthetic clonal reproduction pathway of this invention.

FIG. 2 schematically illustrates the formation of clonal seeds through a combination of formation of diploid gametes with genome elimination.

FIG. 3 illustrates an unrooted NJ 9neighbor-joining) tree of OSD1/UVI4 sequences prepared on-line http://genome.jp using slow/accurate and default parametres. The OSD1 genes in Arabidopsis and rice are each indicated by an arrow.

FIG. 4 provides a schematic comparison of the mechanisms of mitosis, normal meiosis and meiosis in certain mutants as described in the text. The figure is taken from International application WO2010/07943.

FIGS. 5A and B relate to the analysis of cenh3-1 plants as discussed in the Examples. FIG. 5A are illustrations comparing vital staining of pollen grains by Alexander staining of wild-type (1), GFP-tailswap (2), GFP-CENH3 (3), and GFP-CENH3 GFP-tailswap (4). FIG. 5B is a graph summarizing the percentage of viable (black) and dead (grey) pollen from the genotypes indicated.

FIGS. 6A-C provide a summary of the genotype analysis of osd1 [symbol] ×GEM [symbol] (A) and GEM [symbol] ×osd1 [symbol] (B) offspring as discussed in the Examples. FIGS. 6A and 6B summarize the results of genotyping of diploid offspring of the indicated crosses with respect to parental mutations and several trimorphic molecular markers. A color rosace is includes in FIG. 6B that applies to both FIGS. 6A and B. FIG. 6C is a schematic representation of the mechanism of production of diploid uniparental recombined progeny.

FIGS. 7A-C provide a summary of the genotype analysis of MiMe [symbol] ×GEM [symbol] (A), cloned MiMe [symbol] ×GEM [symbol] (B) and GEM [symbol] × [symbol] (C) offspring as discussed in the Examples. Color coding is provided in FIG. 7B which allies to all of FIGS. 7A-C.

### DETAILED DESCRIPTION OF THE INVENTION

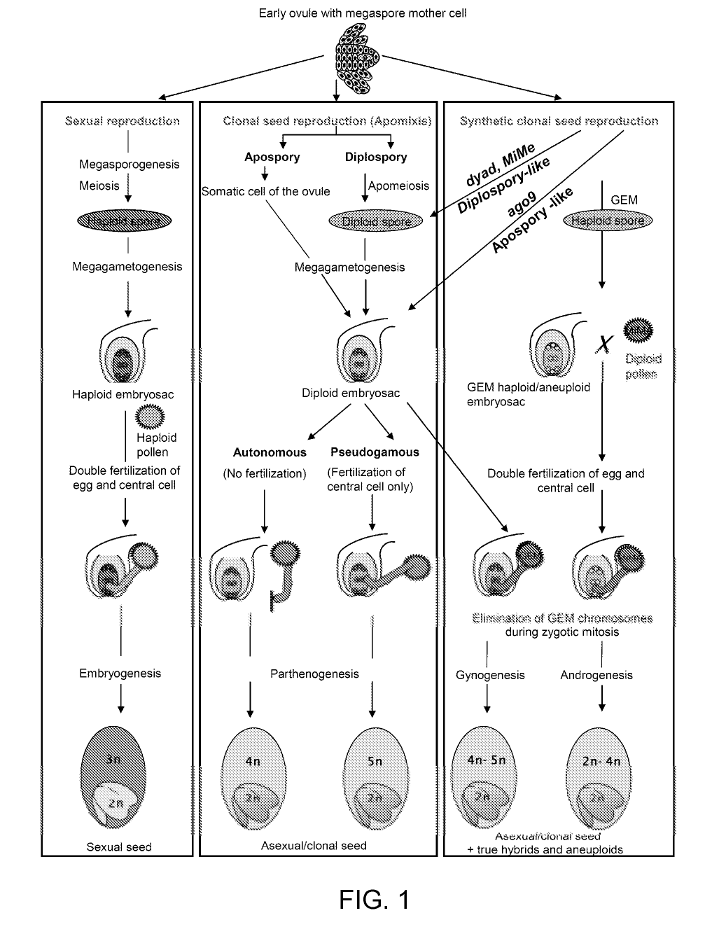

FIG. 1 illustrates an overview of sexual, asexual development and provides a comparison to an exemplary synthetic clonal reproduction pathway of this invention. Nucellar cells of the ovule are plastic and can transdifferentiate to execute different cell fates, leading to either sexual or asexual seed development.

As illustrated in FIG. 1 (left column, sexual development), a subepidermal cell in the early ovule differentiates into an archesporial cell, which at the initiation of meiosis is called the megaspore mother cell (MMC). Sexual development involves three major events:

1) Megasporogenesis: The formation of a megaspore from the archesporial cell of the ovule by meiosis.
2) Megagametogenesis: The formation of an embryo sac (female gametophyte) by the mitotic division of the haploid megaspore.
3) Double fertilization. One sperm cell fuses with the egg cell to form the zygote (2n) and the other sperm cell fertilizes the central cell to form the triploid (3n) embryo nourishing tissue, the endosperm.

As illustrated in FIG. 1 (center column, asexual development-apomixis), the somatic nucellar cell can directly differentiate to form a diploid embryo sac by a process called apospory. Alternatively, in a process called diplospory the MMC can bypass recombination during meiosis and form a diploid spore (apomeiosis). The diploid spore gives rise to a diploid embryo sac. Asexual seed are formed by avoiding fertilization of the diploid egg cell by the male gamete. The diploid egg cell autonomously develops into an embryo (parthenogenesis). The endosperm can develop without fertilization of the central cell (autonomous) or require fertilization of the central cell for normal development (pseudogamous). The ploidy of the endosperm varies depending upon whether the central cell is fertilized or not. Numerous other variations exist for formation of an unreduced megaspore and megagametophyte.

As illustrated in FIG. 1 (right column, synthetic clonal reproduction), MiMe mutants form asexual diploid gametophytes akin to diplosporous apomicts. The clonal egg cell and central cell are then fertilized by pollen of the genome eliminator strain, exemplified by GEM (Genome Elimination caused by a Mix of cenh3 variants, see Examples). In zygotic mitosis, the GEM parental genome is selectively eliminated. The embryo develops solely from the diploid egg cell genome (gynogenesis). In another pathway, GEM haploid embryo sacs are double fertilized by diploid MiMe pollen. After fertilization, the GEM maternal genome is eliminated and the diploid embryo develops from the sperm cell (androgenesis). In either case, the ploidy of endosperm may vary.

Clonal reproduction though seeds is of great interest for agriculture because it allows the propagation of a chosen genotype to the infinite. Endless propagation requires that clonal reproduction can be achieved from generation to generation. As discussed below, the present invention demonstrates that clonal reproduction can be achieved from generation to generation and in principle indefinitely, by crossed a maternal MiMe clone to the exemplary genome eliminator strain GEM for a second generation with the result that the progeny of this cross, produce a large proportion (24%, n=79) of plants genetically identical to their mother and grandmother.

The strategies described herein reflect a de novo synthetic approach to creating apomixis in sexual plants. Given that apomixis in nature occurs by a range of developmental mechanisms it is not unexpected that there would be more than one way of achieving synthetic apomixis. The molecular mechanisms underlying apomixis have resisted elucidation and the genomic regions to which apomixis loci have been mapped are large and show reduced levels of recombination [Ozias-Akins and van Dijk (2007)], making it difficult to identify specific genetic elements that control the trait. It is not unlikely that apomixis as it occurs in nature may be highly context dependent and not readily amenable to transfer to other plant species. The de novo synthesis approach provided herein overcomes this limitation as the genes involved have clear homologues across plant species.

MiMe Plants

A plant having the MiMe (mitosis instead of meiosis) genotype is a plant in which a deregulation of meiosis results in a mitotic-like division and in which meiosis is replaced by mitosis. MiMe plants are exemplified by MiMe-1 plants as described by d'Erfurth, I. et al. (2009) and International patent application WO2001/079432, published Jul. 15, 2010) and MiMe-2 plants as described by d'Erfurth, I. et al. (2010). Each of these three references is incorporated by reference herein in its entirety to provide details of plants having the MiMe genotype and the OSD1 gene and the TAM gene (also designated CYCLIN-A CYCA1;2/TAM, which encodes the Cyclin A CycA1;2 protein) and to provide methods for making MiMe plants. Additional detailed methods provided in these references include sources of plant material, plant growth conditions, genotyping employing PCR and primers useful for such genotyping, and methods of cytology and flow cytometry. These references also provide details of specific mutants employed to produce MiMe plants.

Mercier R. & Grelon M. (2008) provide a recent review of plant meiotic genes which have been functionally characterized, particularly in Arabidopsis, rice and maize. This reference provides an overview of methods employed for such characterization.

Plants having the MiMe genotype produce functional diploid gametes that are genetically identical to their parent. Exemplary MiMe plants combine phenotypes of (1) no second meiotic division, (2) no recombination and (3) modified chromatid segregation.

Exemplary MiMe-1 plants combine inactivation of the OSD1 gene, with the inactivation of two or more other genes, one which encodes a protein necessary for efficient meiotic recombination in plants (e.g., SPO11-1, SPO11-2, PRD1, PRD2, or PAIR1), and whose inhibition eliminates recombination and pairing [Grelon et al., (2001)], and another which encodes a protein necessary for the monopolar orientation of the kinetochores during meiosis, e.g., REC8, and whose inhibition modifies chromatid segregation [Chelysheva et al (2005)]. Exemplary MiMe-2 plants combine inactivation of the TAM gene [d'Erfurth, I. et al. (2010)] with the inactivation of two or more other genes, one which encodes a protein necessary for efficient meiotic recombination in plants (e.g., SPO11-1, SPO11-2, PRD1, PRD2, or PAIR1), and whose inhibition eliminates recombination and pairing [Grelon et al., (2001)], and another which encodes a protein necessary for the monopolar orientation of the kinetochores during meiosis, e.g., REC8, and whose inhibition modifies chromatid segregation [Chelysheva et al (2005)]. MiMe-1 plants are distinguished from MiMe-2 in that MiMe-1 plants are generally more efficient for production of 2N female gametes. For example, in Arabidopsis thaliana specific MiMe-2 mutants generate ˜30% of 2N female gametes, compared to 80% in comparable MiMe-1 mutants [d'Erfurth, I. et al. (2009) and d'Erfurth, I. et al. (2010)].

The replacement of meiosis by mitosis results in apomeiotic gametes, retaining all of the parent's genetic information. The apomeiotic gametes produced by the MiMe mutant can be used, in the same way as SDR (Second Division Restitution) 2n gametes, for producing polyploids plants, or for crossing plants of different ploidy level. They are, however of particularly interest for the production of apomictic plants.

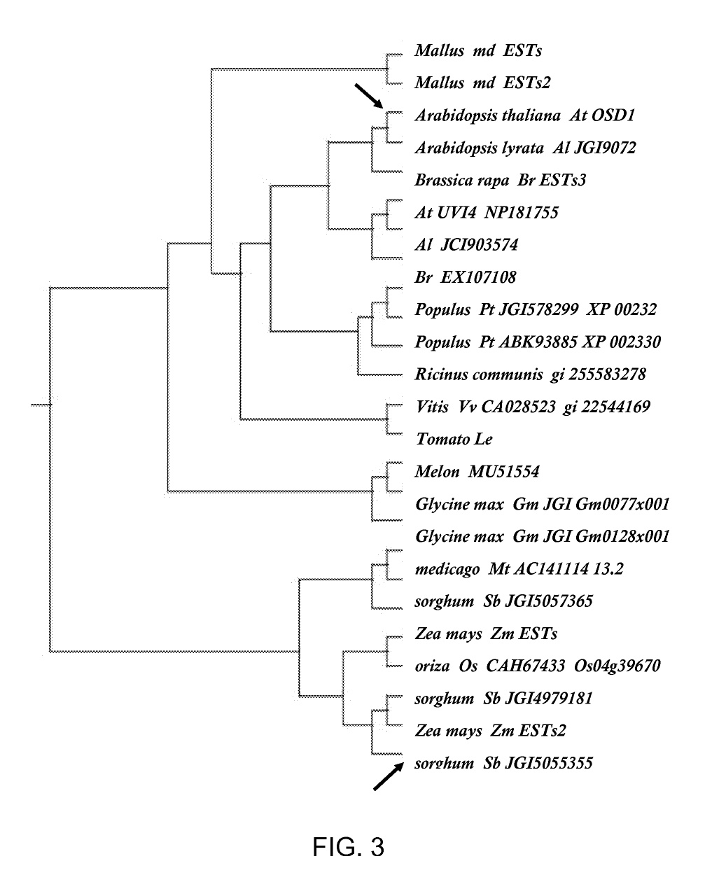

Inactivation of the OSD1 gene (omission of second division) in plants results in the skipping of the second meiotic division. This generates diploid male and female spores, giving rise to viable diploid male and female gametes, which are SDR gametes. The sequence of the OSD1 gene of Arabidopsis thaliana is available in the TAIR database under the accession number At3g57860, or in the GenBank database under the accession number NM_{—}115648. This gene encodes a protein of 243 amino acids (GenBank NP_{—}191345), whose sequence is also represented in the enclosed sequence listing as SEQ ID No. 1, Table 1. The OSD1 gene of Arabidopsis thaliana had previously been designated “UVI4-Like” gene (UVI4-L), which describes its paralogue UVI4 as a suppressor of endo-reduplication and necessary for maintaining the mitotic state (Hase et al. Plant J, 46, 317-26, 2006). However, OSD1 (UVI4-L) does not appear to be required for this process, but is necessary for allowing the transition from meiosis I to meiosis II. An ortholog of the OSD1 gene of Arabidopsis thaliana has been identified in rice (Oryza sativa). The sequence of this gene is available as accession number Os02g37850 in the TAIR database and the gene encodes a protein of 234 amino acid (sequence provided as SEQ ID No. 2, Table 2). The OSD1 proteins of Arabidopsis thaliana and Oryza sativa have 23.6% sequence identity and 35% sequence similarity over the whole length of their sequences. A plant producing Second Division Restitution 2N gametes can, for example, be obtained by inhibition in the plant of an OSD1 protein. Table 13 (SEQ ID Nos. 24-46) provides additional exemplary OSD1/UV14 protein sequences. FIG. 3 includes a list of the OSD1/UV14 protein sequences of Tables 1, 2 and 13 and an NJ (Neighbor-joining) tree of these sequences.

Inactivation of the TAM gene in plants can result in skipping of the second meiotic division giving a phenotype similar to that of osd1 mutants leading to the production of dyads of spores and diploid gametes that have undergone recombination. More specifically, Arabidopsis mutants including tam-2, tam-3, tam-4, tam-5, tam-6 and tam-7 as described in d'Erfurth, I. et al. (2010) express the dyad phenotype at normal growing temperatures and systematically produce mostly dyads. Plant mutants exhibiting inactivation of the TAM gene as in such mutants are useful in preparation of MiMe-2 plants. In contrast, Arabidopsis mutants such as tam-1 [Magnard, J. L. et al. (2001)] which exhibit a delay in the progression of meiosis and progress beyond the dyad stage are not useful in preparation of MiMe-2 plants. The TAM gene encodes a protein exhibiting cyclin-dependent protein kinase activity. The sequence of the TAM gene of Arabidopsis thaliana is available in the TAIR database under the accession number At1 G77390 (Table 9, SEQ ID No. 9). This gene encodes a protein of 442 amino acids (GenBank NP 177863). Cyclin-dependent kinases are reported to be highly conserved among plants and a CycA1;2 gene has been identified in rice (La, H. et al. (2006)]. A Cyclin-A1-2 protein of rice (Accession Q0JPA4-1 in UniProtKB/Swiss-Prot. Database) is identified as having 477 amino acid (Table 10, SEQ ID No. 10). A plant producing Second Division Restitution 2N gametes can, for example, be obtained by inhibition in the plant of an TAM (CycA1;2) protein. Table 12 provides the protein sequence of CYCA1; 2 of A. lyrata (SEQ ID No. 23).

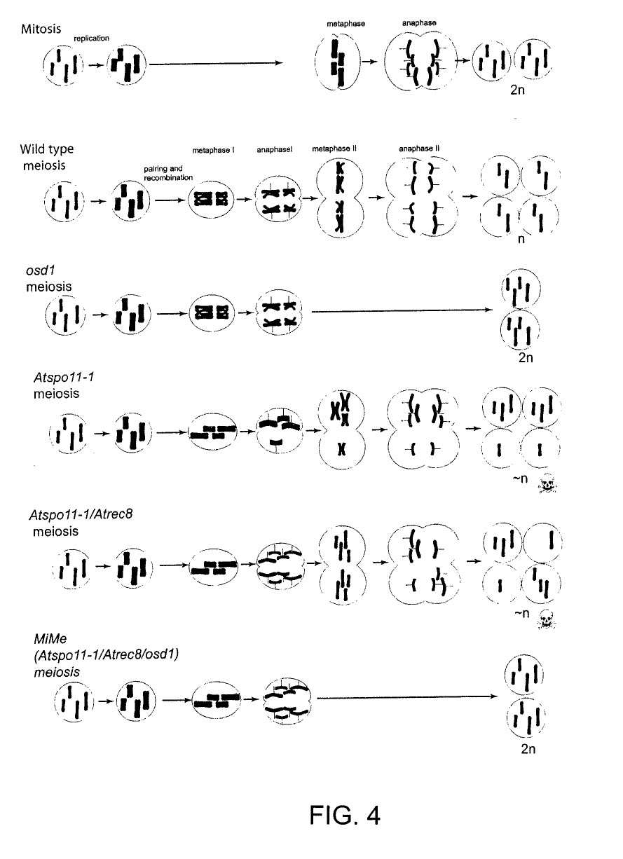

Published International application WO 2010/07943 provides a schematic comparison (reproduced as FIG. 4 herein) between the mechanisms of mitosis, normal meiosis, meiosis in an osd1 mutant, meiosis in a mutant lacking SPO11-1 activity (e.g., Atspo11-1), meiosis in a double mutant lacking both SPO11-1 and REC8 activity (e.g., Atspo11-1/Atrec8), and meiosis in a MiMe mutant (e.g., osd1/Atspo11-1/Atrec8). During mitosis in diploid cells, chromosomes replicate and sister chromatids segregate to generate daughter cells that are diploid and genetically identical to the initial cell. During normal meiosis, two rounds of chromosome segregation follow a single round of replication. At division one, homologous chromosomes recombine and are separated. Meiosis II is more similar to mitosis resulting in equal distribution of sister chromatids. The spores obtained are thus haploid and carry recombined genetic information. In a mutant lacking OSD1 activity, meiosis II is skipped giving rise to diploid spores and SDR gametes with recombined genetic information. A mutant lacking SPO11-1 undergoes an unbalanced first division followed by a second division leading to unbalanced spores and sterility. A double mutant lacking both SPO11-1 and REC8 undergoes a mitotic-like division instead of a normal first meiotic division, followed by an unbalanced second division leading to unbalanced spores and sterility. Arabidopsis MiMe-2 mutants are described in d'Erfurth, I. et al. (2010)

SPO11-1 and SPO11-2 proteins are related orthologs, both of which are required for meiotic recombination. [Grelon et al. (2001); Stacey et al. (2006); Hartung et al. (2007)]. Inhibition of one or both of SPO11-1 or SPO11-2 is useful in a MiMe plant of this invention. Examples of SPO11-1 and SPO11-2 proteins are provided in Table 3 (SEQ ID No. 3) and Table 4 (SEQ ID No. 4).

PRD1 protein is required for meiotic double stand break (DSB) formation and is exemplified by AtPRD1, a protein of 1330 amino acids (Table 5, SEQ ID No. 5) exhibiting significant sequence similarity with OsPRD1 (NCB1 Accession number CAE02100) SEQ ID No. 47 (Table 14). PRD1 homologs have also been identified in Physcomitrella patens (PpPRD1) from ASYA488561.b1; Medicago truncatula (MtPRD1) from sequences AC147484 (start 93451-end 101276) and Populus trichocarpa (PtPRD1) from LG_II:20125180-20129370 (http://genome.jgi-psf.org/Poptr1_{—}1/Poptr1_{—}1.home.html), see De Muyt et al. 2007, FIG. 1 therein for a sequence comparison.

PRD2 protein is a DSB-forming protein exemplified by AtPRD2, a protein of 378 amino acids (Table 6, SEQ ID No: 6) amino acids (identified as a protein of 385 amino acids in De Muyt et al. (2009) see Sequence Accession NP 568869 (Table 11, SEQ ID No. 18), with homologues identified in the monocot Oryza sativa, Populous trichocarpa, Vitis vinifera and Physcomitrella patens [De Muyt et al. (2009)] and see (Table 11, SEQ ID Nos. 19-22). PAIR1 (also called PRD3) is a DSB-forming protein exemplified by AtPAIR1, a protein a 449 amino acid protein (Table 7, SEQ ID No. 7) and its presumed ortholog OsPAIR1 [Nonomura et al. (2004)] a 492-amino acid protein, see Table 15, SEQ ID No. 50.

REC8 protein is a subunit of the cohesion complex. In plants, exemplified by Arabidopsis, REC8 protein (Table 8, SEQ ID No. 8) is necessary for monopolar orientation of the kinetochores [Chelysheva et al. (2005)].

In specific embodiments, plants producing apomeiotic gametes are produced by inhibition in the plant of the following proteins (a) a TAM (Cylin A CYCA1;2) protein (as described herein); (b) a protein involved in initiation of meiotic recombination in plants exemplified herein as SPO11-1; SPO11-2; PRD; PRD2; or PAIR1 (also called PRD3); and (c) a protein necessary for the monopolar orientation of the kinetochores during meiosis exemplified herein as REC8 protein.

In specific embodiments, plants producing apomeiotic gametes are produced by inhibition in the plant of the following proteins (a) an OSD 1 protein (as described herein); (b) a protein involved in initiation of meiotic recombination in plants exemplified herein as SPO11-1; SPO11-2; PRD; PRD2; or PAIR1 (also called PRD3); and (c) a protein necessary for the monopolar orientation of the kinetochores during meiosis exemplified herein as REC8 protein.

The OSD1 protein is exemplified by the AtOSD1 protein (SEQ ID No. 1) or the Os OSD1 protein (SEQ ID No. 2) and includes OSD1 protein wherein said protein has at least 20%, and by order of increasing preference, at least 25, 30, 35, 40, 45, 50, 55, 60, 65, 70, 75, 80, 85, 90, 95 or 98% sequence identity, or at least 29%, and by order of increasing preference, at least 35, 40, 45, 50, 55, 60, 65, 70, 75, 80, 85, 90, 95 or 98% sequence similarity with the AtOSD1 protein of SEQ ID No. 1 or with the OsOSD1 protein of SEQ ID No. 2.

The Cyclin-A CYCA1;2 (TAM) protein is exemplified by the CYCA1; 2 protein of Arabidopsis (SEQ ID No. 9) or the CYCA1; 2 protein of rice (SEQ ID No. 10) protein wherein said protein has at least 20%, and by order of increasing preference, at least 25, 30, 35, 40, 45, 50, 55, 60, 65, 70, 75, 80, 85, 90, 95 or 98% sequence identity, or at least 29%, and by order of increasing preference, at least 35, 40, 45, 50, 55, 60, 65, 70, 75, 80, 85, 90, 95 or 98%

The protein involved in initiation of meiotic recombination in plants is exemplified by an SPO11-1 or SPO11-2 protein and particularly the AtSPO11-1 protein (SEQ ID No. 3), the AtSPO11-2 protein (SEQ ID No. 4) and includes SPO11-1 and SPO11-2 proteins having at least 40%, and by order of increasing preference, at least 45, 50, 55, 60, 65, 70, 75, 80, 85, 90, 95 or 98% sequence identity, or at least 60%, and by order of increasing preference, at least, 65, 70, 75, 80, 85, 90, 95 or 98% sequence similarity with the SPO11-1 protein of SEQ ID No. 3 or the SPO11-2 protein of SEQ ID No. 4.

The protein involved in initiation of meiotic recombination in plants is also exemplified by a PRD1 or PRD2 protein and particularly the AtPRD1 protein (SEQ ID No. 5), and the AtPRD2 protein (SEQ ID No. 6) and includes PRD1 or PRD2 proteins having at least 25%, and by order of increasing preference, at least 30, 35, 40, 45, 50, 55, 60, 65, 70, 75, 80, 85, 90, 95 or 98% sequence identity, or at least 35%, and by order of increasing preference, at least, 40, 45, 50, 55, 60, 65, 70, 75, 80, 85, 90, 95 or 98% sequence similarity with the PRD1 protein of SEQ ID No. 5) or PRD2 protein of SEQ ID No. 6).

The protein involved in initiation of meiotic recombination in plants is also exemplified by a PAIR1 protein (also known as a PRD3 protein) and particularly the AtPAIR1 protein (SEQ ID No. 7), and includes PAIR1 proteins having at least 30%, and by order of increasing preference, at least 35, 40, 45, 50, 55, 60, 65, 70, 75, 80, 85, 90, 95 or 98% sequence identity, or at least 40%, and by order of increasing preference, at least, 45, 50, 55, 60, 65, 70, 75, 80, 85, 90, 95 or 98% sequence similarity with the PAIR1 protein of SEQ ID No. 7.

The protein necessary for the monopolar orientation of the kinetochores during meiosis is exemplified herein as a REC8 protein (also designated DIF1/SYN1) a member of the cohesion complex in plants, particularly Arabidopsis. REC8 protein includes AtREC8 protein (SEQ ID No. 8) and includes REC8 protein having at least 40%, and by order of increasing preference, at least 45, 50, 55, 60, 65, 70, 75, 80, 85, 90, 95 or 98% sequence identity, or at least 45%, and by order of increasing preference at least, 50, 55, 60, 65, 70, 75, 80, 85, 90, 95 or 98% sequence similarity with the REC8 protein of SEQ ID No. 8.

The SPO11-1, SPO11-2, PRD1, PRD2, PAIR1, and REC8 proteins are conserved in higher plants, monocotyledons as well as dicotyledons. By way of non-limitative examples of orthologs of SPO11-1, SPO11-2, PRD1, PRD2, PAIR1 and REC8 proteins of Arabidopsis thaliana in monocotyledonous plants, one can cite the Oryza sativa SPO11-1, SPO11-2, PRD1, PRD2, PAIR1, and REC8 proteins. The sequence of the Oryza sativa SPO11-1 protein is available in GenBank under the accession number AAP68363 see Table 15 SEQ ID No. 48; the sequence of the Oryza sativa SPO11-2 protein is available in GenBank under the accession number NP_{—}001061027 see Table 15 SEQ ID No. 49; the sequence of the Oryza sativa PRD1 protein is provided as SEQ ID No. 47 (Table 14); the sequence of the Oryza sativa PRD2 protein is provided (SEQ ID No. 21); the sequence of the Oryza sativa PAIR1 protein is available in SwissProt under the accession number Q75RY2, see Table 15 SEQ ID No. 50; the sequence of the Oryza sativa REC8 protein (also designated RAD21-4) is available in GenBank under the accession number AAQ75095., see Table 15, SEQ ID No. 51. Additional non-limiting examples of orthologs of PRD2 include Vitis vinifera VvPRD2 (accession number CAO66652) see Table 11, SEQ ID No. 20; Populous trichocarpa PtPRD2 (obtained from JCI (fgenesh4_pm.C_LG_VI000547) see Table 11 SEQ ID NO. 20 and Physcomitrella patens PpPRD2 obtained from JGI (jgi|Phypa1_{—}1|73600|fgenesh1_pg.scaffold_{—}42000158).

The inhibition of the above mentioned OSD1, Cyclin-A CYCA1;2 (TAM), SPO11-1, SPO11-2, PRD1, PRD2, PAIR1, or REC8 proteins can be obtained either by abolishing, blocking, or decreasing their function, or advantageously, by preventing or down-regulating the expression of the corresponding genes. By way of example, inhibition of said protein can be obtained by mutagenesis of the corresponding gene or of its promoter, and selection of the mutants having partially or totally lost the activity of said protein. For instance, a mutation within the coding sequence can induce, depending on the nature of the mutation, the expression of an inactive protein, or of a protein with impaired activity; in the same way, a mutation within the promoter sequence can induce a lack of expression of said protein, or decrease thereof.

Mutagenesis can be performed for instance by targeted deletion of the coding sequence or of the promoter of the gene encoding said protein or of a portion thereof, or by targeted insertion of an exogenous sequence within said coding sequence or said promoter. It can also be performed by inducing random mutations, for instance through EMS mutagenesis or random insertional mutagenesis, followed by screening of the mutants within the desired gene. Methods for high throughput mutagenesis and screening are available in the art. By way of example, one can mention TILLING (Targeting Induced Local Lesions In Genomes) described by McCallum et al., 2000).

Among the mutations within the OSD1 gene or TAM gene, those resulting in the ability to produce SDR 2n gametes can be identified on the basis of the phenotypic characteristics of the plants which are homozygous for this mutation: these plants can form at least 5%, preferably at least 10%, more preferably at least 20%, yet more preferably 30% or more, still more preferably at least 50%, and up to 100% of dyads as a product of meiosis.

Among the mutations within a gene encoding a protein involved in initiation of meiotic recombination in plants, such as the SPO11-1 gene or the SPO11-2, PRD1, PRD2 or PAIR1 gene, those useful for obtaining a plant producing apomeiotic gametes can be identified on the basis of the phenotypic characteristics of the plants which are homozygous for this mutation, in particular the presence of univalents instead of bivalents at meiosis I, and the sterility of the plant. Among the mutants having a mutation within the REC8 gene, those useful for obtaining a plant producing apomeiotic gametes can be identified on the basis of the phenotypic characteristics of the plants which are homozygous for this mutation, in particular chromosome fragmentation at meiosis, and sterility of the plant.

Alternatively, the inhibition of the target protein is obtained by silencing of the corresponding gene. [See, for example, the review Baulcombe, D. (2004)]. Methods for gene silencing in plants are known in the art. For instance, antisense inhibition or co-suppression, as described by way of example in U.S. Pat. Nos. 5,190,065 and 5,283,323 can be used. It is also possible to use ribozymes targeting the mRNA of said protein. Preferred methods are those wherein gene silencing is induced by means of RNA interference (RNAi), using a silencing RNA targeting the gene to be silenced. Various methods and DNA constructs for delivery of silencing RNAs are available in the art.

A “silencing RNA” is herein defined as a small RNA that can silence a target gene in a sequence-specific manner by base pairing to complementary mRNA molecules. Silencing RNAs include in particular small interfering RNAs (siRNAs) and microRNAs (miRNAs).

Initially, DNA constructs for delivering a silencing RNA in a plant included a fragment of 300 bp or more (generally 300-800 bp, although shorter sequences may sometime induce efficient silencing) of the cDNA of the target gene, under transcriptional control of a promoter active in said plant. Currently, the more widely used silencing RNA constructs are those that can produce hairpin RNA (hpRNA) transcripts. In these constructs, the fragment of the target gene is inversely repeated, with generally a spacer region between the repeats [for a review, see Watson et al., (2005)]. One can also use artificial microRNAs (amiRNAs) directed against the gene to be silenced (for review about the design and applications of silencing RNAs, including in particular amiRNAs, in plants see for instance [Ossowski et al., (2008)].

Tools for silencing one or more target gene(s) selected among OSD1, TAM, SPO11-1 SPO11-2, PRD1, PAIR1, PRD2, and REC8, including expression cassettes for hpRNA or amiRNA targeting said gene (s) are described and provided in PCT application WO 2010/079432. Useful expression cassettes comprise a promoter functional in a plant cell; one or more DNA construct(s) of 200 to 1000 bp, preferably of 300 to 900 bp, each comprising a fragment of a cDNA of a target gene selected among OSD1, TAM, SPO11-1, SPO11-2, PRD1, PRD2, PAIR1, and REC8, or of its complement, or having at least 95% identity, and by order of increasing preference, at least 96%, 97%, 98%, or 99% identity with said fragment, where the DNA construct(s) is placed under transcriptional control of the promoter. Additional useful expression cassettes for hpRNA comprise a promoter functional in a plant cell, one or more hairpin DNA construct(s) capable, when transcribed, of forming a hairpin RNA targeting a gene selected among OSD1, TAM, SPO11-1, SPO11-2, PRD1, PRD2, PAIR1, and REC8; where the DNA construct(s) is placed under transcriptional control of the promoter.

Generally, useful hairpin DNA constructs comprise: i) a first DNA sequence of 200 to 1000 bp, preferably of 300 to 900 bp, such as a fragment of a cDNA of the target gene, or having at least 95% identity, and by order of increasing preference, at least 96%, 97%, 98%, or 99% identity with the fragment; ii) a second DNA sequence that is the complement of the first DNA, said first and second sequences being in opposite orientations and ii) a spacer sequence separating the first and second sequence, such that these first and second DNA sequences are capable, when transcribed, of forming a single double-stranded RNA molecule. The spacer can be a random fragment of DNA. However, preferably, one will use an intron which is spliceable by the target plant cell. Its size is generally 400 to 2000 nucleotides in length. A useful expression cassette for an amiRNA comprises: a promoter functional in a plant cell, one or more DNA construct(s) capable, when transcribed, of forming an amiRNA targeting a gene selected among OSD1, TAM, SPI11-1, SPO11-2, PRD1, PRD2, PAIR1, and REC8; where the DNA construct(s) is placed under transcriptional control of the promoter. Useful expression cassettes comprise a DNA construct targeting the OSD1 gene or comprise a DNA construct targeting the OSD1 gene, and a DNA construct targeting a gene selected from one or more of SPO11-1, SPO11-2, PRD1, PRD2, or PAIR1, and a DNA construct targeting REC8. Useful expression cassettes comprise a DNA construct targeting the TAM gene or comprise a DNA construct targeting the TAM gene, and a DNA construct targeting a gene selected from one or more of SPO11-1, SPO11-2, PRD1, PRD2, or PAIR1, and a DNA construct targeting REC8. Additional useful expression cassettes comprise a DNA construct targeting the OSD1 gene and/or the TAM gene and/or comprise a DNA construct targeting the OSD1 gene and or the TAM gene, and/or a DNA construct targeting a gene selected from one or more of SPO11-1, SPO11-2, PRD1, PRD2, or PAIR1.

It will be appreciated by one of ordinary skill in the art that a large choice of promoters suitable for expression of heterologous genes in plants is available in the art. Useful promoters include those obtained from plants, plant viruses, or bacteria, such as Agrobacterium. Promoters include constitutive promoters, i.e. promoters which are active in most tissues and cells and under most environmental conditions, as well as tissue-specific or cell-specific promoters which are active only or mainly in certain tissues or certain cell types, and inducible promoters that are activated by physical or chemical stimuli, such as those resulting from nematode infection. Non-limiting examples of constitutive promoters that are commonly used in plant cells are the cauliflower mosaic virus (CaMV) 35S promoter, the Nos promoter, the rubisco promoter, or the Cassava vein Mosaic Virus (CsVMV) promoter. Organ or tissue specific promoters that can be used in such expression cassettes include in particular promoters able to confer meiosis-associated expression, such as the DMC1 promoter [Klimyuk & Jones (1997)]; one can also use any of the endogenous promoters of the genes OSD1, TAM, SPO11-1, SPO11-2, PRD1, PRD2, PAIR1, or REC8. Useful DNA constructs of the invention generally also include a transcriptional terminator (for instance the 35S transcriptional terminator, or the nopaline synthase (Nos) transcriptional terminator).

Recombinant vectors, host cells comprising recombinant DNA constructs, transgenic plants, transgenic plant cells and methods of transforming plants with a vector targeting the OSD1 gene and/or the TAM gene and/or a vector targeting one or more of the SPO11-1, SPO11-2, PRD1, PRD2, or PAIR1 genes and/or a vector targeting the REC8 gene and regenerating such transgenic plants are described and provided in PCT application WO 2010/079432 and are useful in preparation of MiMe plants useful in this invention. The expression of a chimeric DNA construct targeting the OSD1 gene, and which results in a down regulation of the OSD1 protein, provides to a transgenic plant the ability to produce 2n SDR gametes. The expression of a chimeric DNA construct targeting the TAM gene, and which results in a down regulation of the Cyclin A CycA1;2 protein, provides to a transgenic plant the ability to produce 2n SDR gametes. The co-expression of a chimeric DNA construct targeting the OSD1 gene and/or the TAM gene, a chimeric DNA construct targeting a gene selected among one or more of SPO11-1, SPO11-2, PRD1, PRD2 and PAIR1, and a chimeric DNA construct targeting the REC8 gene and which results in down regulation of the proteins encoded by these genes provides to a transgenic plant the ability to produce apomeiotic gametes. MiMe plants include those which produce at least 10%, more preferably at least 20%, and by order of increasing preference, at least 30%, 40%, 50%, or 60%, 70%, 80%, or 90% of viable apomeiotic gametes. MiMe plants also include those that are heterozygous for the MiMe.

The genes discussed above which confer the MiMe genotype are strongly conserved among plants, including monocots and dicots, thus, the MiMe genotype can be engineered, for example, as described herein in any plant species, including crop species. In specific embodiments, the MiMe genotype can be engineered as described herein in various species of Arabidopsis, in various crop plants including without limitation, rice, soybean, corn or maize, rye, cotton, oats, barley, wheat, alfalfa, sorghum, sunflower, various legumes, various Brassica, potato, peanuts, clover, sweet potato, cassava (manioc), rye-grass, banana, melon, watermelon, sugar beets, sugar cane, lettuce, carrots, spinach, endive, leeks, celery, artichokes, beets, radishes, turnips or tomato or ornamental plants such as roses, lilies, tulips or narcissus.

MiMe plants of this invention can be further engineered employing techniques that are well known to one of ordinary skill in the art to contain one or more non-endogenous genes or mutated endogenous genes the expression of which provides: (1) one or more gene products useful for screening or selection of such plants; or (2) one or more agriculturally useful traits. Methods of the present invention allow generation of clonal embryos or seeds which will retain such one or more non-endogenous genes or mutated genes.

Genome Eliminator Strains

Haploid inducer plants with directed genome elimination have been identified, generated or engineered in various plants and in particular in maize and Arabidopsis. Plants which induce genome elimination as described herein function for genome elimination in crossings with any MiMe plant.

U.S. Pat. No. 5,749,169 describes certain haploid inducer maize plants which induce genome elimination (ig plants-indeterminate gametophyte), including homozygous (igig) plants which can be used to generate androgenetic haploids. Female ig plants are pollinated with pollen from a selected maize plant, e.g., one carrying a mutation associated with a desirable phenotype. Progeny from such crosses include a significantly enhanced percentage of androgenetic haploids containing chromosomes derived only from the male parent. Maize ig plants exhibiting approximately 1 to 3% androgenetic haploids of total progeny are reported. Maize ig plants induce haploids of both male and female origin. The ig trait was initially reported as arising in the inbred Wisconsin-23 (W23) strain (Kermicle, J. L., 1969). U.S. Pat. No. 5,749,169 is incorporated by reference herein for its description of haploid inducers, particularly in maize and for methods of making and identifying such haploid inducers.

U.S. Pat. No. 5,639,951 describes maize haploid inducers, particularly those exhibiting the ig genotype and having a least one dominant gene which may be a conditional lethal gene, a screenable marker gene or a selectable marker gene. The presence of the dominant gene is useful in screening and selection methods. U.S. Pat. No. 5,639,951 is incorporated by reference herein for its description of haploid inducers with dominant genes as described, particularly in maize, and for methods of making an identifying such haploid inducers.

Maize genotypes which induce gynogenesis producing maternal haploids with chromosomes derived from the female parent have been described. Such inducer lines for maize include, but are not limited to, Stock 6 and Stock 6 derivatives [Coe, (1959); Sarkar & Coe, (1966); Sarkar et al. (1972), Lashermes & Beckert (1988), Chalyk, S. T. (1994), Bordes, J. R. et al. (1997), Eder J. & Chalyk, S. (2002) RWS [Röber et al. (2005)], KEMS [Deimling, et al. (1997)], or KMS and ZMS [Chalyk, S. T. et al. (1994), Chalyk & Chebotar (2000)]. The Stock 6 derivative WS14 [Lashermes & Beckert (1988)] is reported to exhibit haploid induction rate that is 1.2 to 5.5 times higher than that of Stock 6. A WS14 derivative designated FIGH 1 [Bordes et al. (1997)] is also of interest. Crosses between two haploid-inducing lines can be used generate progeny haploid inducers exhibiting higher rates of haploid induction compared to their parents, for examples crosses between KMS and ZMS lines are reported to be capable of inducing 7 to 9% of haploids [Chalyk et al. (1994)]. The disclosure of each of the foregoing references is incorporated by reference herein in its entirety for its description of haploid inducer lines, methods for identifying and/or making such lines, and sources of material for making such lines.

International patent application WO 2005/004586 describes certain gynogenetic haploids in maize which are designated as in the PK6 line of maize or derivative lines thereof. Haploid inducers of this maize line are reported to exhibit rates of gynogenetic haploid induction much superior to those observed with prior art haploid inducers. WO 2005/004586 is incorporated by reference herein in its entirety for descriptions of PK6 plants and derivatives thereof as well as for methods of making such plants by breeding and/or transformation methods.

Geiger H. H. & Gordillo (2009) provide a description of measurement of haploid induction rates and provide examples of maize haploid inducer lines (e.g., RWS, RWK-76 and the cross RWS×RWK-76) having higher haploid inducer rates (e.g., greater than 1%). This reference is incorporated by reference herein for details of the measurement of haploid induction rate and for sources of haploid inducers having higher haploid inducer rates.

Genome eliminator strains of this invention include all such haploid inducers and derivatives thereof. Haploid inducers include derivatives of the specifically mentioned haploid inducers which are generated by conventional plant breeding methods.

Mutants Having Altered CENH3 Protein

Mutants having altered CENH3 protein are exemplified by those described in Ravi, M & Chan, S. W-L. 2010 and Ravi, M. et al. Jul. 13, 2010. Each of which references is incorporated by reference in its entirety herein for description of such mutants and methods for making such mutants. Published patent application US 2011/0083202 A1 (Chan and Maruthachalam, Apr. 7, 2011) provides description of altered CENH3 protein and is incorporated by reference herein in its entirety for that description.

It will be appreciated however that CENH3 variants other than those specifically described in Ravi, M & Chan, S. W-L. 2010 and Ravi, M. et al. Jul. 13, 2010 are useful for making genome eliminator plants of this invention. It will be appreciated for example that useful CENH3 variants for a given plant can be obtained by replacing the N-terminal tail domain of the CENH3 endogenous in that plant with the N-terminal tail domain of a centromere specific histone of the same species of plant or that of a different species of plant or that of another organism.

It will be appreciated that any GFP-tag in an altered variant of CENH3 can be replaced with various other known tags (e.g., β-galactosidase, cyan fluorescent protein (CYP), or yellow fluorescent protein (YFP)) by methods that are well known in the art. Thus, tagged-CENH3 variants are useful in the methods of this invention.

Additional altered CENH3 useful in this invention preferably exhibits overall % identity of amino acid sequence to the endogenous CENH3 that is at least 25% and by order of increasing preference, at least 30, 35, 40, 45, 50, 55, 60, 65, 70, 75, 80, 85, 90, 95 or 98%, or at least 35%, and by order of increasing preference at least, 40, 45, 50, 55, 60, 65, 70, 75, 80, 85, 90, 95 or 98% overall sequence similarity to the endogenous CENH3.

In specific embodiments, altered CENH3 having a GFP tag or functionally equivalent other tag (e.g., β-galactosidase, cyan fluorescent protein (CYP), yellow fluorescent protein (YFP, e.g., PhiYFP (Trademark, Evrogen)) can exhibit overall % identity of amino acid sequence to the endogenous CENH3 that is at least 50% and by order of increasing preference, at least 55, 60, 65, 70, 75, 80, 85, 90, 95, 96, or 98%, or at least 60%, and by order of increasing preference at least, 65, 70, 75, 80, 85, 90, 95, 96 or 98% overall sequence similarity to the endogenous CENH3.

Further additional altered CENH3 useful in this invention preferably exhibit % identity of amino acid sequence to the histone fold region of the endogenous CENH3 that is at least 50% and by order of increasing preference, at least 55, 60, 65, 70, 75, 80, 85, 90, 95, 96 or 98%, or at least 60%, and by order of increasing preference at least, 65, 70, 75, 80, 85, 90, 95, 96 or 98% sequence similarity to the histone fold region of the endogenous CENH3.

In specific embodiments, altered CENH3 having a GFP tag or functionally equivalent other tag, can exhibit overall % identity of amino acid sequence to the histone fold region of endogenous CENH3 that is at least 50% and by order of increasing preference, at least 55, 60, 65, 70, 75, 80, 85, 90, 95, 96, or 98%, or at least 60%, and by order of increasing preference at least, 65, 70, 75, 80, 85, 90, 95, 96 or 98% overall sequence similarity to the histone fold region of endogenous CENH3.

Plants expressing one, two or more altered CENH3 proteins which are haploid inducers preferably exhibit haploid induction rates of 1% or more and by order of increasing preference, 3% or more, 5% or more, 10% or more, 20% or more or 30% or more.

It will be appreciated that transformant plants expressing altered CENH3 may exhibit differences in expression level caused by position effects. One of ordinary skill in the art knows how to detect such position effects which may affect expression levels of altered CENH3 protein and select transformants with expression levels which exhibit levels of expression of one, two or more altered CENH3 protein that provide for haploid induction.

Useful CENH3 variants can be prepared by methods as described in Ravi, M & Chan, S. W-L. 2010 and Ravi, M. et al. Jul. 13, 2010 employing expression cassettes and plant transformation methods as described therein or by any means know in the art which would be appreciated by one of ordinary skill in the art to provide for expression of such variants in plants.

It will be appreciated that plants expressing CENH3 variants useful as haploid inducers can be prepared in various plants including without limitation in both monocots or dicots. Plants expressing such altered CENH3 genotypes can be engineered, for example, as described herein in any plant species, including crop species. In specific embodiments, the altered CENH3 genotype can be engineered as described herein in various species of Arabidopsis, in various crop plants including without limitation, rice, soybean, corn or maize, rye, cotton, oats, barley, wheat, alfalfa, sorghum, sunflower, various legumes, various Brassica, potato, peanuts, clover, sweet potato, cassava (manioc), rye-grass, banana, melon, watermelon, sugar beets, sugar cane, lettuce, carrots, spinach, endive, leeks, celery, artichokes, beets, radishes, turnips or tomato or ornamental plants such as roses, lilies, tulips or narcissus.

Unless otherwise specified, the protein sequence identity and similarity values provided herein are calculated over the whole length of the sequences, using the BLASTP program under default parameters, or the Needleman-Wunsch global alignment algorithm (EMBOSS pairwise alignment Needle tool under default parameters). Similarity calculations are performed using the scoring matrix BLOSUM62.

As used herein, the term “plant” includes reference to whole plants, plant organs (e.g., leaves, stems, roots, etc.), seeds and plant cells and progeny of same. “Plant cell”, as used herein includes, without limitation, seeds, suspension cultures, embryos, meristematic regions, callus tissue, leaves, roots, shoots, gametophytes, sporophytes, pollen, and microspores.

MiMe plants or any of the various haploid inducer plants useful in this invention can include, or be bred or engineered to include and express a selectable or screenable marker gene. Selectable markers generally include genes encoding antibiotic resistance or resistance to herbicide, which are known in the art. Screenable markers include β-galactosidase, green fluorescent protein (GFP), cyan fluorescent protein (CYP), yellow fluorescent protein (YFP, e.g., PhiYFP (Trademark, Evrogen)). MiMe plants or any of the various haploid inducer plants useful in this invention can include, or be bred or engineered to include and express a gene or combination of genes conveying a phenotype or trait of interest, such a phenotype or trait of agricultural interest. Conventional plant breeding methods or plant transformation methods may be used to generate such derivatives of MiMe plants and/or haploid inducer plants.

A portion of the subject matter of this application is reported in Marimuthu M. P et al. 2011, which is incorporated by reference herein in its entirety.

When a grouping is used herein, all individual members of the group and all possible combinations and subcombinations of the members of the groups therein are intended to be individually included in the disclosure. Every plant mutant, line or strain, or combination thereof described or exemplified herein can be used to practice the invention, unless otherwise stated.

One of ordinary skill in the art will appreciate that methods, procedures and materials, such as methods for detecting the presence or absence of genes or proteins, hybridization methods, PCR methods, culturing methods and media, other than those specifically exemplified herein can be employed in the practice of the invention without resort to undue experimentation. All art-known functional equivalents, of any such methods, materials and conditions are intended to be included in this invention.

Whenever a range is given in the specification, for example, a range of numbers, a range of any integer, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the broad term “comprising”, particularly in a description of components of a composition, the recitation of steps in a method or in a description of elements of a device, is intended to encompass and describe the terms “consisting essentially of” or “consisting of”.

Although the description herein contains many specific details, these should not be construed as limiting the scope of the invention, but as merely providing illustrations of some of the embodiments of the invention. Each patent document and publication referenced in this specification is incorporated by reference herein to the same extent as if each individual document or publication was specifically and individually indicated to be incorporated by reference. In the case of any inconsistency between the content of a cited reference and the disclosure herein, the disclosure of this specification is to be given priority. Some references cited herein are incorporated by reference herein to provide details of haploid inducers and methods of making such haploid inducers, methods for making and mutants useful for making MiMe plants, methods for crossing specified plants, hybridization methods for the detection of genes, other methods for the detection of expression of certain genes in plants, PCR methods for the detection of expression of certain genes, methods for generating CENH3 variants, assay conditions, particularly hybridization assay conditions and PCR assay conditions, additional methods of analysis and additional uses of the invention.

TABLE 1
Arabidopsis thaliana At
OSD1(NP_191345)(SEQ ID No 1):

MPEARDRTERPVDYSTIFANRRRHGILLDEPDSRLSLIESPVNPDIGSIG
GTGGLVRGNFTTWRPGNGRGGHTPFRLPQGRENMPIVTARRGRGGGLLPS
WYPRTPLRDITHIVRAIERRRGAGTGGDDGRVIEIPTHRQVGVLESPVPL
SGEHKCSMVTPGPSVGFKRSCPPSTAKVQKMLLDITKEIAEEEAGFITPE
KKLLNSIDKVEKIVMAEIQKLKSTPQAKREEREKRVRTLMTMR

TABLE 2
Oriza osOSD1 Os02g37850
Os|BAD17434 (SEQ ID No. 2):

MPEVRNSGGRAALADPSGGGFFIRRTTSPPGAVAVKPLARRALPPTSNKE
NVPPSWAVTVRATPKRRSPLPEWYPRSPLRDITSVVKAVERKSRLGNAAV
RQQIQLSEDSSRSVDPATPVQKEEGVPQSTPTPPTQKALDAAAPCPGSTQ
AVASTSTAYLAEGKPKASSSSPSDCSFQTPSRPNDPALADLMEKELSSSI
EQIEKMVRKNLKRAPKAAQPSKVTIQKRTLLSMR

TABLE 3
Arabidopsis thaliana SPO11-1 (SEQ ID No. 3):

Met Glu Gly Lys Phe Ala Ile Ser Glu Ser Thr Asn
Leu Leu Gln Arg Ile Lys Asp Phe Thr Gin Ser Val
Val Val Asp Leu Ala Glu Gly Arg Ser Pro Lys Ile
Ser Ile Asn Gln Phe Arg Asn Tyr Cys Met Asn Pro
Glu Ala Asp Cys Leu Cys Ser Ser Asp Lys Pro Lys
Gly Gln Glu Ile Phe Thr Leu Lys Lys Glu Pro Gln
Thr Tyr Arg Ile Asp Met Leu Leu Arg Val Leu Leu
Ile Val Gln Gln Leu Leu Gln Glu Asn Arg His Ala
Ser Lys Arg Asp Ile Tyr Tyr Met His Pro Ser Ala
Phe Lys Ala Gln Ser Ile Val Asp Arg Ala Ile Gly
Asp Ile Cys Ile Leu Phe Gln Cys Ser Arg Tyr Asn
Leu Asn Val Val Ser Val Gly Asn Gly Leu Val Met
Gly Trp Leu Lys Phe Arg Glu Ala Gly Arg Lys Phe
Asp Cys Leu Asn Ser Leu Asn Thr Ala Tyr Pro Val
Pro Val Leu Val Glu Glu Val Glu Asp Ile Val Ser
Leu Ala Glu Tyr Ile Leu Val Val Glu Lys Glu Thr
Val Phe Gln Arg Leu Ala Asn Asp Met Phe Cys Lys
Thr Asn Arg Cys Ile Val Ile Thr Gly Arg Gly Tyr
Pro Asp Val Ser Thr Arg Arg Phe Leu Arg Leu Leu
Met Glu Lys Leu His Leu Pro Val His Cys Leu Val
Asp Cys Asp Pro Tyr Gly Phe Glu Ile Leu Ala Thr
Tyr Arg Phe Gly Ser Met Gln Met Ala Tyr Asp Ile
Glu Ser Leu Arg Ala Pro Asp Met Lys Trp Leu Gly
Ala Phe Pro Ser Asp Ser Glu Val Tyr Ser Val Pro
Lys Gin Cys Leu Leu Pro Leu Thr Glu Glu Asp Lys
Lys Arg Thr Glu Ala Met Leu Leu Arg Cys Tyr Leu
Lys Arg Glu Met Pro Gln Trp Arg Leu Glu Leu Glu
Thr Met Leu Lys Arg Gly Val Lys Phe Glu Ile Glu
Ala Leu Ser Val His Ser Leu Ser Phe Leu Ser Glu
Val Tyr Ile Pro Ser Lys Ile Arg Arg Glu Val Ser
Ser Pro

TABLE 4
Arabidopsis thaliana SPO11-2 9SEQ ID No. 4):

Met Glu Glu Ser Ser Gly Leu Ser Ser Met Lys Phe
Phe Ser Asp Gln His Leu Ser Tyr Ala Asp Ile Leu
Leu Pro His Glu Ala Arg Ala Arg Ile Glu Val Ser
Val Leu Asn Leu Leu Arg Ile Leu Asn Ser Pro Asp
Pro Ala Ile Ser Asp Leu Ser Leu Ile Asn Arg Lys
Arg Ser Asn Ser Cys Ile Asn Lys Gly Ile Leu Thr
Asp Val Ser Tyr Ile Phe Leu Ser Thr Ser Phe Thr
Lys Ser Ser Leu Thr Asn Ala Lys Thr Ala Lys Ala
Phe Val Arg Val Trp Lys Val Met Glu Ile Cys Phe
Gln Ile Leu Leu Gln Glu Lys Arg Val Thr Gln Arg
Glu Leu Phe Tyr Lys Leu Leu Cys Asp Ser Pro Asp
Tyr Phe Ser Ser Gln Ile Glu Val Asn Arg Ser Val
Gln Asp Val Val Ala Leu Leu Arg Cys Ser Arg Tyr
Ser Leu Gly Ile Met Ala Ser Ser Arg Gly Leu Val
Ala Gly Arg Leu Phe Leu Gln Glu Pro Gly Lys Glu
Ala Val Asp Cys Ser Ala Cys Gly Ser Ser Gly Phe
Ala Ile Thr Gly Asp Leu Asn Leu Leu Asp Asn Thr
Ile Met Arg Thr Asp Ala Arg Tyr Ile Ile Ile Val
Glu Lys His Ala Ile Phe His Arg Leu Val Glu Asp
Arg Val Phe Asn His Ile Pro Cys Val Phe Ile Thr
Ala Lys Gly Tyr Pro Asp Ile Ala Thr Arg Phe Phe
Leu His Arg Met Ser Thr Thr Phe Pro Asp Leu Pro
Ile Leu Val Leu Val Asp Trp Asn Pro Ala Gly Leu
Ala Ile Leu Cys Thr Phe Lys Phe Gly Ser Ile Gly
Met Gly Leu Glu Ala Tyr Arg Tyr Ala Cys Asn Val
Lys Trp Ile Gly Leu Arg Gly Asp Asp Leu Asn Leu
Ile Pro Glu Glu Ser Leu Val Pro Leu Lys Pro Lys
Asp Ser Gln Ile Ala Lys Ser Leu Leu Ser Ser Lys
Ile Leu Gln Glu Asn Tyr Ile Glu Glu Leu Ser Leu
Met Val Gln Thr Gly Lys Arg Ala Glu Ile Glu Ala
Leu Tyr Cys His Gly Tyr Asn Tyr Leu Gly Lys Tyr
Ile Ala Thr Lys Ile Val Gln Gly Lys Tyr Ile

TABLE 5
Arabidopsis thaliana PRD1 sequence (SEQ ID No. 5):

Met Phe Phe Gln His Ser Gln Leu Gln Asn Ser Asp His Leu Leu His
Glu Ser Met Ala Asp Ser Asn His Gln Ser Leu Ser Pro Pro Cys Ala
Asn Gly His Arg Ser Thr Ile Ser Leu Arg Asp Asp Gln Gly Gly Thr
Phe Cys Leu Ile Cys Phe Ser Asn Leu Val Ser Asp Pro Arg Ile Pro
Thr Val His Val Ser Tyr Ala Leu His Gln Leu Ser Ile Ala Ile Ser
Glu Pro Ile Phe Leu Arg Thr Leu Leu Ser Ser His Ile His Phe Leu
Val Ser Pro Leu Val His Ala Leu Ser Ser Ile Asp Asp Ala Pro Ile
Ala Ile Gln Ile Met Asp Met Ile Ser Leu Leu Cys Ser Val Glu Glu
Ser Ser Ile Gly Glu Asp Phe Val Glu Arg Ile Ser Asp Gln Leu Ser
Ser Gly Ala Leu Gly Trp Ser Arg Arg Gln Leu His Met Leu His Cys
Phe Gly Val Leu Met Ser Cys Glu Asn Ile Asp Ile Asn Ser His Ile
Arg Asp Lys Glu Ala Leu Val Cys Gln Leu Val Glu Gly Leu Gln Leu
Pro Ser Glu Glu Ile Arg Gly Glu Ile Leu Phe Ala Leu Tyr Lys Phe
Ser Ala Leu Gln Phe Thr Glu Gln Asn Val Asp Gly Ile Glu Val Leu
Ser Leu Leu Cys Pro Lys Leu Leu Cys Leu Ser Leu Glu Ala Leu Ala
Lys Thr Gln Arg Asp Asp Val Arg Leu Asn Cys Val Ala Leu Leu Thr
Ile Leu Ala Gln Gln Gly Leu Leu Ala Asn Ser His Ser Asn Ser Ala
Ser Ser Met Ser Leu Asp Glu Val Asp Asp Asp Pro Met Gln Thr Ala
Glu Asn Val Ala Ala Arg Pro Cys Leu Asn Val Leu Phe Ala Glu Ala
Ile Lys Gly Pro Leu Leu Ser Thr Asp Ser Glu Val Gln Ile Lys Thr
Leu Asp Leu Ile Phe His Tyr Ile Ser Gln Glu Ser Thr Pro Ser Lys
Gln Ile Gln Val Met Val Glu Glu Asn Val Ala Asp Tyr Ile Phe Glu
Ile Leu Arg Leu Ser Glu Cys Lys Asp Gln Val Val Asn Ser Cys Leu
Arg Val Leu Asp Leu Phe Ser Leu Ala Glu His Ser Phe Arg Lys Arg
Leu Val Ile Gly Phe Pro Ser Val Ile Arg Val Leu His Tyr Val Gly
Glu Val Pro Cys His Pro Phe Gln Ile Gln Thr Leu Lys Leu Ile Ser
Ser Cys Ile Ser Asp Phe Pro Gly Ile Ala Ser Ser Ser Gln Val Gln
Glu Ile Ala Leu Val Leu Lys Lys Met Leu Glu Arg Tyr Tyr Ser Gln
Glu Met Gly Leu Phe Pro Asp Ala Phe Ala Ile Ile Cys Ser Val Phe
Val Ser Leu Met Lys Thr Pro Ser Phe Gly Glu Thr Ala Asp Val Leu
Thr Ser Leu Gln Glu Ser Leu Arg His Ser Ile Leu Ala Ser Leu Ser
Leu Pro Glu Lys Asp Ser Thr Gln Ile Leu His Ala Val Tyr Leu Leu
Asn Glu Ile Tyr Val Tyr Cys Thr Ala Ser Thr Ser Ile Asn Met Thr
Ser Cys Ile Glu Leu Arg His Cys Val Ile Asp Val Cys Thr Ser His
Leu Leu Pro Trp Phe Leu Ser Asp Val Asn Glu Val Asn Glu Glu Ala
Thr Leu Gly Ile Met Glu Thr Phe His Ser Ile Leu Leu Gln Asn Ser
Asp Ile Gln Ala Lys Glu Phe Ala Glu Leu Leu Val Ser Ala Asp Trp
Phe Ser Phe Ser Phe Gly Cys Leu Gly Asn Phe Cys Thr Asp Asn Met
Lys Gln Arg Ile Tyr Leu Met Leu Ser Ser Leu Val Asp Ile Leu Leu
Glu Gln Lys Thr Gly Ser His Ile Arg Asp Ala Leu His Cys Leu Pro
Ser Asp Pro Gln Asp Leu Leu Phe Leu Leu Gly Gln Ala Ser Ser Asn
Asn Gln Glu Leu Ala Ser Cys Gln Ser Ala Ala Leu Leu Ile Phe His
Thr Ser Ser Ile Tyr Asn Asp Arg Leu Ala Asp Asp Lys Leu Val Leu
Ala Ser Leu Glu Gln Tyr Ile Ile Leu Asn Lys Thr Ser Leu Ile Cys
Ala Ile Ser Asp Ser Pro Ala Leu Leu Asn Leu Val Asn Leu Tyr Gly
Leu Cys Arg Ser Leu Gln Asn Glu Arg Tyr Gln Ile Ser Tyr Ser Leu
Glu Ala Glu Arg Ile Ile Phe His Leu Leu Asn Glu Tyr Glu Trp Asp
Leu Gly Ser Ile Asn Ile His Leu Glu Ser Leu Lys Trp Leu Phe Gln
Gln Glu Ser Ile Ser Lys Ser Leu Ile Tyr Gln Ile Gln Lys Ile Ser
Arg Asn Asn Leu Ile Gly Asn Glu Val His Asn Val Tyr Gly Asp Gly
Arg Gln Arg Ser Leu Thr Tyr Trp Phe Ala Lys Leu Ile Ser Glu Gly
Asp Asn Tyr Ala Ala Thr Leu Leu Val Asn Leu Leu Thr Gln Leu Ala
Glu Lys Glu Glu Gln Glu Asn Asp Val Thr Ser Ile Leu Asn Leu Met
Asn Thr Ile Val Ser Ile Phe Pro Thr Ala Ser Asn Asn Leu Ser Met
Asn Gly Ile Gly Ser Val Val His Arg Leu Val Ser Gly Phe Ser Asn
Ser Ser Leu Gly Thr Ser Phe Lys Thr Leu Leu Leu Leu Val Phe Asn
Ile Leu Thr Ser Val Gln Pro Ala Val Leu Met Ile Asp Glu Ser Trp
Tyr Ala Val Ser Ile Lys Leu Leu Asn Phe Leu Ser Leu Arg Asp Thr
Ala Ile Lys Gln Asn His Glu Asp Met Val Val Ile Gly Ile Leu Ser
Leu Val Leu Tyr His Ser Ser Asp Gly Ala Leu Val Glu Ala Ser Arg
Asn Ile Val Ser Asn Ser Tyr Leu Val Ser Ala Ile Asn Thr Val Val
Asp Val Ala Cys Ser Lys Gly Pro Ala Leu Thr Gln Cys Gln Asp Glu
Thr Asn Ile Gly Glu Ala Leu Ala Phe Thr Leu Leu Leu Tyr Phe Phe
Ser Leu Arg Ser Leu Gln Ile Val Leu Ala Gly Ala Val Asp Trp
Gln Ala Phe Phe Gly Thr Ser Thr Ser Leu Glu Thr Leu Pro Val
Val Cys Ile Tyr Cys His Asn Leu Cys Arg Leu Met His Phe Gly
Ala Pro Gln Ile Lys Leu Ile Ala Ser Tyr Cys Leu Leu Glu Leu
Leu Thr Gly Leu Ser Glu Gln Val Asp Ile Lys Lys Glu Gln Leu
Gln Cys Ser Ser Ser Tyr Leu Lys Ser Met Lys Ala Val Leu Gly
Gly Leu Val Phe Cys Asp Asp Ile Arg Val Ala Thr Asn Ser Ala
Leu Cys Leu Ser Met Ile Leu Gly Trp Glu Asp Met Glu Gly Arg
Thr Glu Met Leu Lys Thr Ser Ser Trp Tyr Arg Phe Ile Ala Glu
Glu Met Ser Val Ser Leu Ala Leu Pro Cys Ser Ala Ser Ser Thr
Tyr Val Asn His His Lys Pro Ala Val Tyr Leu Thr Val Ala Met
Leu Arg Leu Lys Asn Lys Pro Val Trp Leu Arg Thr Val Phe Asp
Glu Ser Cys Ile Ser Ser Met Ile Gln Asn Leu Asn Gly Ile Asn
Ile Ser Arg Glu Ile Val Ile Leu Phe Arg Glu Leu Met Gln Ala
Glu Leu Leu Asn Ser Gln Gln Val Thr Lys Leu Asp Arg Ala Phe
Gln Glu Cys Arg Lys Gln Met His Arg Asn Gly Thr Arg Asp Glu
Thr Val Glu Glu Gln Val Gln Arg Lys Ile Pro Ser Ile His Asp
His Ser Glu Phe Cys Asn Tyr Leu Val His Leu Met Val Ser Asn
Ser Phe Gly His Pro Ser Glu Ser Glu Thr Tyr Thr Gln Lys Lys
Lys Gln Ile Leu Asp Glu Met Glu Gln Phe Ser Glu Leu Ile Ser
Thr Arg Glu Gly Arg Val Ser Pro Ile Gln Glu Glu Thr Arg Gln
Met Gln Thr Glu Arg Ile Val

TABLE 6
Arabidopsis thaliana gi|260590345|emb|CAX83745.1|
putative recombination initiation defect 2
protein (SEQ ID NO: 6):

MSSSVAEANHTEKEESLRLAIAVSLLRSKFHNHQSSSSTSRCYVSSESD
ALRWKQKAKERKKEIIRLQEDLKDAESSFHRDLFPANASCKCYFFDNLG
VFSGRRIGEASESRFNDVLRRRFLRLARRRSRRKLTRSSQRLQPSEPDY
EEEAEHLRISIDFLLELSEADSNDSNFSNWSHQAVDFIFASLKKLISMG
RNLESVEESISFMITQLITRMCTPFKGNEVKQLETSVGFYVQHLIRKLG
SEPFIGQRAIFAISQRISILAENLLFMDPFDESFPEMDECMFILIQLIE
FLICDYLLPWAENEAFDNVMFEEWIASVVHARKAVKALEERNGLYLLYM
DRVTGELAKRVGQITSFREVEPAILDKILAYQEIE

TABLE 7
Arabidopsis thaliana PAIR1 (SEQ ID No. 7):

Met Lys Met Asn Ile Asn Lys Ala Cys Asp Leu Lys Ser Ile Ser Val
Phe Pro Pro Asn Leu Arg Arg Ser Ala Glu Pro Gln Ala Ser Gln Gln
Leu Arg Ser Gln Gln Ser Gln Gln Ser Phe Ser Gln Gly Pro Ser Ser
Ser Gln Arg Gly Cys Gly Gly Phe Ser Gln Met Thr Gln Ser Ser Ile
Asp Glu Leu Leu Ile Asn Asp Gln Arg Phe Ser Ser Gln Glu Arg Asp
Leu Ser Leu Lys Lys Val Ser Ser Cys Leu Pro Pro Ile Asn His Lys
Arg Glu Asp Ser Gln Leu Val Ala Ser Arg Ser Ser Ser Gly Leu Ser
Arg Arg Trp Ser Ser Ala Ser Ile Gly Glu Ser Lys Ser Gln Ile Ser
Glu Glu Leu Glu Gln Arg Phe Gly Met Met Glu Thr Ser Leu Ser Arg
Phe Gly Met Met Leu Asp Ser Ile Gln Ser Asp Ile Met Gln Ala Asn
Arg Gly Thr Lys Glu Val Phe Leu Glu Thr Glu Arg Ile Gln Gln Lys
Leu Thr Leu Gln Asp Thr Ser Leu Gln Gln Leu Arg Lys Glu Gln Ala
Asp Ser Lys Ala Ser Leu Asp Gly Gly Val Lys Phe Ile Leu Glu Glu
Phe Ser Lys Asp Pro Asn Gln Glu Lys Leu Gln Lys Ile Leu Gln Met
Leu Thr Thr Ile Pro Glu Gln Val Glu Thr Ala Leu Gln Lys Ile Gln
Arg Glu Ile Cys His Thr Phe Thr Arg Glu Ile Gln Val Leu Ala Ser
Leu Arg Thr Pro Glu Pro Arg Val Arg Val Pro Thr Ala Pro Gln Val
Lys Ala Lys Glu Asn Leu Pro Glu Gln Arg Gly Gln Ala Ala Lys Val
Leu Thr Ser Leu Lys Met Pro Glu Pro Arg Val Gln Val Pro Ala Ala
Pro Gln Ala Lys Glu Asn Phe Pro Glu Gln Arg Gly Pro Val Ala Lys
Ser Asn Ser Phe Cys Asn Thr Thr Leu Lys Thr Lys Gln Pro Gln Phe
Pro Arg Asn Pro Asn Asp Ala Ser Ala Arg Ala Val Lys Pro Tyr Leu
Ser Pro Lys Ile Gln Val Gly Cys Trp Lys Thr Val Lys Pro Glu Lys
Ser Asn Phe Lys Lys Arg Ala Thr Arg Lys Pro Val Lys Ser Glu Ser
Thr Arg Thr Gln Phe Glu Gln Cys Ser Val Val Ile Asp Ser Asp Glu
Glu Asp Ile Asp Gly Gly Phe Ser Cys Leu Ile Asn Glu Asn Thr Arg
Gly Thr Asn Phe Glu Trp Asp Ala Glu Lys Glu Thr Glu Arg Ile Leu
Arg Thr Ala Arg Arg Thr Lys Arg Lys Phe Gly Asn Pro Ile Ile Ile
Asn

TABLE 8
Arabidopsis thaliana REC8 (SEQ ID No. 8):

Met Phe Tyr Ser His Gln Leu Leu Ala Arg Lys Ala Pro Leu Gly Gln
Ile Trp Met Ala Ala Thr Leu His Ala Lys Ile Asn Arg Lys Lys Leu
Asp Lys Leu Asp Ile Ile Gln Ile Cys Glu Glu Ile Leu Asn Pro Ser
Val Pro Met Ala Leu Arg Leu Ser Gly Ile Leu Met Gly Gly Val Val
Ile Val Tyr Glu Arg Lys Val Lys Leu Leu Phe Asp Asp Val Asn Arg
Phe Leu Val Glu Ile Asn Gly Ala Trp Arg Thr Lys Ser Val Pro Asp
Pro Thr Leu Leu Pro Lys Gly Lys Thr His Ala Arg Lys Glu Ala Val
Thr Leu Pro Glu Asn Glu Glu Ala Asp Phe Gly Asp Phe Glu Gln Thr
Arg Asn Val Pro Lys Phe Gly Asn Tyr Met Asp Phe Gln Gln Thr Phe
Ile Ser Met Arg Leu Asp Glu Ser His Val Asn Asn Asn Pro Glu Pro
Glu Asp Leu Gly Gln Gln Phe His Gln Ala Asp Ala Glu Asn Ile Thr
Leu Phe Glu Tyr His Gly Ser Phe Gln Thr Asn Asn Glu Thr Tyr Asp
Arg Phe Glu Arg Phe Asp Ile Glu Gly Asp Asp Glu Thr Gln Met Asn
Ser Asn Pro Arg Glu Gly Ala Glu Ile Pro Thr Thr Leu Ile Pro Ser
Pro Pro Arg His His Asp Ile Pro Glu Gly Val Asn Pro Thr Ser Pro
Gln Arg Gln Glu Gln Gln Glu Asn Arg Arg Asp Gly Phe Ala Glu Gln
Met Glu Glu Gln Asn Ile Pro Asp Lys Glu Glu His Asp Arg Pro Gln
Pro Ala Lys Lys Arg Ala Arg Lys Thr Ala Thr Ser Ala Met Asp Tyr
Glu Gln Thr Ile Ile Ala Gly His Val Tyr Gln Ser Trp Leu Gln Asp
Thr Ser Asp Ile Leu Cys Arg Gly Glu Lys Arg Lys Val Arg Gly Thr
Ile Arg Pro Asp Met Glu Ser Phe Lys Arg Ala Asn Met Pro Pro Thr
Gln Leu Phe Glu Lys Asp Ser Ser Tyr Pro Pro Gln Leu Tyr Gln Leu Trp Ser
Lys Asn Thr Gln Val Leu Gln Thr Ser Ser Ser Glu Ser Arg His Pro Asp Leu
Arg Ala Glu Gln Ser Pro Gly Phe Val Gln Glu Arg Met His Asn His His Gln Thr
Asp His His Glu Arg Ser Asp Thr Ser Ser Gln Asn Leu Asp Ser Pro Ala Glu Ile
Leu Arg Thr Val Arg Thr Gly Lys Gly Ala Ser Val Glu Ser Met Met Ala Gly Ser
Arg Ala Ser Pro Glu Thr Ile Asn Arg Gln Ala Ala Asp Ile Asn Val Thr Pro Phe
Tyr Ser Gly Asp Asp Val Arg Ser Met Pro Ser Thr Pro Ser Ala Arg Gly Ala Ala Ser
Ile Asn Asn Ile Glu Ile Ser Ser Lys Ser Arg Met Pro Asn Arg Lys Arg Pro Asn Ser
Ser Pro Arg Arg Gly Leu Glu Pro Val Ala Glu Glu Arg Pro Trp Glu His Arg Glu Tyr
Glu Phe Glu Phe Ser Met Leu Pro Glu Lys Arg Phe Thr Ala Asp Lys Glu Ile Leu
Phe Glu Thr Ala Ser Thr Gln Thr Gln Lys Pro Val Cys Asn Gln Ser Asp Glu Met Ile
Thr Asp Ser Ile Lys Ser His Leu Lys Thr His Phe Glu Thr Pro Gly Ala Pro Gln Val Glu
Ser Leu Asn Lys Leu Ala Val Gly Met Asp Arg Asn Ala Ala Ala Lys Leu Phe Phe Gln
Ser Cys Val Leu Ala Thr Arg Gly Val Ile Lys Val Asn Gln Ala Glu Pro Tyr Gly Asp Ile
Leu Ile Ala Arg Gly Pro Asn Met

TABLE 9
Arabidopsis thaliana ACCESSION NP_177863 442 aa
(CYCLIN A1; 2); cyclin-dependent protein kinase
regulator(SEQ ID NO: 9):

MSSSSRNLSQENPIPRPNLAKTRTSLRDVGNRRAPLGDITNQKNGSRNPS
PSSTLVNCSNKIGQSKKAPKPALSRNWNLGILDSGLPPKPNAKSNIIVPY
EDTELLQSDDSLLCSSPALSLDASPTQSDPSISTHDSLTNHVVDYMVEST
TDDGNDDDDDEIVNIDSDLMDPQLCASFACDIYEHLRVSEVNKRPALDYM
ERTQSSINASMRSILIDWLVEVAEEYRLSPETLYLAVNYVDRYLTGNAIN
KQNLQLLGVTCMMIAAKYEEVCVPQVEDFCYITDNTYLRNELLEMESSVL
NYLKFELTTPTAKCFLRRFLRAAQGRKEVPSLLSECLACYLTELSLLDYA
MLRYAPSLVAASAVFLAQYTLHPSRKPWNATLEHYTSYRAKHMEACVKNL
LQLCNEKLSSDVVAIRKKYSQHKYKFAAKKLCPTSLPQELFL

TABLE 10
OsCYCLIN-A1-2 (Q0JPA4 UniProtKB CCA12_ORYSJ)
(SEQ ID NO: 10):

MAAKRPAAGE GGGKAAAGAA AAKKRVALVN ITNVAAAANN
AKFNSATWAA PVKKGSLASG RNVCTNRVSA VKSASAKPAP
AISRHESAPQ KESVIPPKVL SIVPTAAPAP VTVPCSSFVS
PMHSGDSVSV DETMSMCDSM KSPDFEYIDN GDSSSVLGSL
QRRANENLRI SEDRDVEETK WNKDAPSPME IDQICDVDNN
YEDPQLCATL ASDIYMHLRE AETRKRPSTD FMETIQKDVN
PSMRAILIDW LVEVAEEYRL VPDTLYLTVN YIDRYLSGNE
INRQRLQLLG VACMLIAAKY EEICAPQVEE FCYITDNTYF
RDEVLEMEAS VLNYLKFEVT APTAKCFLRR FVRVAQVSDE
DPALHLEFLA NYVAELSLLE YNLLSYPPSL VAASAIFLAK
FILQPTKHPW NSTLAHYTQY KSSELSDCVK ALHRLFSVGP
GSNLPAIREK YTQHKKFVAK KHCPPSVPSE FFRDATC

TABLE 11
Plant PRD2 SEQUENCES

Arabidopsis thaliana ACCESSION (NP_568869) (385 aa) [DeMuyt et al.
(2009] (SEQ ID NO. 18):
MSSSVAEANHTEKEESLRLAIAVSLLRSKFQNHQSSSSTSRCYVSSESDALRWK
QKAKERKKEIIRLQEDLKDAESSFHRDLFPANASCKCYFFDNLGVFSGRRIGEASE
SRFNDVLRRRFLRLACVVILSLARRRSRRKLTRSSQRLQPSEPDYEEEAEHLRISID
FLLELSEADSNDSNFSNWSHQAVDFIFASLKKLISMGRNLESVEESISFMITQLITRM
CTPVKGNEVKQLETSVGFYVQHLIRKLGSEPFIGQRAIFAISQRISILAENLLFMDPFD
ESFPEMDECMFILIQLIEFLICDYLLPWANEAFDNVMFEEWIASVVHARKAVKALEER
NGLYLLYMDRVTGELAKRVGQITSFREVEPAILDKILAYQEIE
Populus trichocarpa gi|224091813|ref|XP_002309357.1 (SEQ ID NO. 19):
MASSEPATDTKTASSPTDDQSLKLAVAISLLRSKLLQKQPPPPPPPSNPPSES
DALRWKRKAKERKQELLRLREDLREAEDASQCDLFPQTALCKCYFFDNLGKS
SPKPVGDGSDRRFNDILRRRFLRQVRIKERRKRINNSNIKIRFSDIYSKNEAEQL
RAAVDFLVELCDTTSPGRVEEANFANWSHQAADFILASLRNLLSIGNNMELIEGI
VSRLIVRLVKRMCSPSHGDESRQTDTDTQFYIQQLIRKLGCEPHIGQRAILSVSQ
RISMVAENLLFLDPFDEAFSNMHECLFIMIQLIEFLISDYLLTWSRDEGFDHVLFEE
WVTSVLHARKALELLESRNGLYVLYMDRVTGELAKHVGQVSSFQKLSQDILDNLF
Vitis vinifera gi|225445826|ref|XP_002275398.1| (SEQ ID NO. 20):
MSTSNTDSHQSLKLAVAMALLRSKLLHNTNPPPPHSDALRWKRKAKERKQELL
RLKEDLREAEDGLRHDLFPPSASCKCHFFDDLGKLSPNQFERGSNRNFNDVLR
RRFLRQVRLKERRRKRTDDSIKHNHYSDIVCEDETEQLRASIDFLVELCDTASPN
SNFTNWSHQAVDFILASLKNLLSVRKNVEYIKGIINSLIKHLVRRLCTPLKGDELHH
LDADHQFYVQHLIRKLGSDPFVGHRAILSVSQRISLIAESLLFLDPFDDAFPNLHGC
MFVLIQLIEFLISDYFLVWSRDEGFDNMLFVEWVTSILHARKALELLESRNGLYVLY
MDRVTGELAKHVGQVSLLQELNPDIINILFH
Oryza sativa Japonica Group gi|297608983|ref|NP_001062471.2|
Os08g0555800 SEQ ID No. 21):
MAPPASRPPTPTPTPTANAAASSSRIESPSLRAALAMALIHYNRLPSRAAAAAA
PSPQALLNWKRKAKDRKREILRLREELKLLQDGARGEEMEPPVASCRCHFFDG
CGDLPPPTDGDAGEHWVDDVLRRRFVRLEYNTEDEVQQLSLSIDFLVELSDGLF
AKREAGSSFTTFSHQAVDFILASLKNILSSEREKEIIEEIINGLVARLMKRMCTTPEN
AGSVDCSDAQFSLQHLFRKLGNEEFVGQRIILAISQKISNVSEKLLLADPFDDGFPE
MHSNMFIMIQLIEFLISDSFNNWLCRDHFDRKLFEEWVRSILKARKDLEVLDGRNGL
YVVYIERVIGRLAREVAPAAHQGKLDLEDGSTMWSMRYLRPHEAIELATSTDSPCIL
VIGGCLPLFVSPTKKEKKEALDSTARCFASLLA
Zea mays gi|212275736|ref|NP_001130070.1| LOC100191163 (SEQ ID No. 22):
MALPKPRPPTPTASAATGTSSSRIDSPSLKAALAMALIHYNRLPGKANATAGTS
PPSLLHWKRKAKDRKREILRLREELKVLQDGVRGEEMEPPVASCRCHFFDGCR
DLRPQQGGGGGEHWVDEVLRRRFLRLVRWKEKRRRVDRSLPSSSLIDFNSEDE
MQQLSMSTDFLVELSDGIFAKSEAGHSFATFSHQAVDFILATLKNILSSEREKDLVG
EIIDSLVTRLMKRMCTVPEKLVTSDSGSTGCSDAQFSVQHLFRKLGNDEFFGQRVIL
VVSQKISNVSERLFLADPFADAFPDMHDNIFIMIQLLEFLISDYMKVWLCCEHINKRLF
EECTRSILKARNDLQILENMNGLYVVYIERVVGRLARDVAPAAHQGKLDLEVFSKLLC

TABLE 12
Arabidopsis lyrata subsp. lyrata ACCESSION
XP_002889141 443 aa CYCA1_2 (SEQ ID No. 23):

MSSSSSSKNLSQENPIPRPNLAKTRTSLRDVGNRRVPLGDITNQKTGSR
NSSSSSTLVHCSNKISQSKKASKPALSRNWNLGILDCGLPPKSNANSNI
IVPYEDTELPQIDDSLLSSSPGLSVDASPTHSDPSISTHDSLKSHIVEH
MVESSTDDGNDDDEIVNIDSDLMDPQLCASFAFDIYEHLRASEVKKRPA
LDYMERIQLNINASMRSILIDWLVEVAEEYRLSPETLYLAVNYVDRYLT
GNAINKQNLQLLGVACMMIAAKYEEVCVPQVEDFCYITDNTYLRNELLE
MESSVLNYLKFELTTPTAKCFLRRFLRAAQGRKEVPSLLSECLACYLTE
LSLLDYMLRYAPSLVAASAVFLAQYILHPSRKPWNATLEHYTSYRAKHM
EACVKNLLQLCNEKPSSDVVAIRKKYSQHKYKFAAKKLCPTSLPQELFLC

TABLE 13
Exemplary OSD1 Protein Sequences

Arabidopsis lyrata Al JGI907257 XP_002876442
(SEQ ID No. 24):
MPEARDRIERPVDYPAIFVNRRSNGVLLDEPDSRLSLIESPVNPETGSMG
RGSLVGTGGLVRGNFSTWRPGNGRGGHSPFRLSQGRENNMPMVSARRGRG
PSLLPSWYPRTPLRDITHIMRTIERRRGAGIGGDDGRDIEIPTHQQVGVL
ESPVPLSGEHKCSIVTPGPSVGFKRSCPPSTAKVHKMLLDITKEIAEEEA
GFITPEKKLLNSIDKVEKIVMAEIQKLKSTPHAKREEREKRVRTLMSMR
Brassica rapa Br ESTs3 (SEQ ID No. 25):
MAEARDRLEKPVDYAAIFANRRSHGVLLDEPEAGLGVLEHPVRRLPSGSR
VYPQPGGNYSSWRPGHGNGSGQSPFRFSQGRENVTMASARRGRGGASGSL
LPSWYPRTPLRDITHIMRAIERKRRAGMGVESALGGETPSHQQVRFLETP
VALAEDEHNCVMVTPAPAVGLKRSCPPSTAKVHKMLLDITKDISDNDEQA
RFITPEKKLLNSIDVVEKIVMAEIQKLKSTPLAKRQEREKRVKTLMSMR
Arabidopsis thanliana UVI4 NP_181755 (SEQ ID No.
26):
MPEARDRIERQVDYPAAFLNRRSHGILLDEPATQHNLFGSPVQRVPSEAT
GGLGSIGQGSMTGRGGLVRGNFGIRRTGGGRRGQIQFRSPQGRENMSLGV
TRRGRARASNSVLPSWYPRTPLRDISAVVRAIERRRARMGEGVGRDIETP
TPQQLGVLDSLVPLSGAHLEHDYSMVTPGPSIGFKRPWPPSTAKVHQILL
DITRENTGEEDALTPEKKLLNSIDKVEKVVMEEIQKMKSTPSAKRAEREK
RVRTLMSMR
Arabidopsis lyrata Al JGI903574 (SEQ ID No. 27):
MPEARDRIERPVDYPAAFLNRRSHGILLDEPATHHNLFGSPVQRVPSEAT
GLGSVGQGSMMGRGGLVRGNFGIRRTGGGRRGQIQFRSPQGRENMSLGVT
RRGRARASNSVLPSWYPRTPLRDVSAVVRAVERRRARMGEGVGRDIETPT
PQQLGVLDSLVPLSGAQLEHDYSMVTPGPSVGFKRPWPPSTAKVHQILLD
ITRENTGEEDALTPQKKLLNSIDKVEKVVMEEIQKMKSTPSAKRAEREKR
VRTLMSMR
Brassica rapa EX107108 (SEQ ID No. 28):
MPEARDRRERSVDYPAAFLNRRSHGILLDESPLRSPVQRLPSSESLVFGR
GGFARGNLGIRRTGGGGGRRRGRARASASVLPSWYPRTPLRDVSSVVRAI
ERRRARVGDVETPTPQQLEVVLDDSLAPVSGERNYSMVTPGPSVGFKRPW
PPSTAKVHQILLDITRQSSAEEEEEALTPQKKLLNSIDKVEKVVMEEIQK
MKSTPSAKRAEREKRVRTLMSMR
Populus Pt JGI576299 XP_002323297 (SEQ ID No. 29):
MTESRDRLSRAVDIAAIFAARRQSMNLGIYQDRPELDMALFGSPRTNTAI
RNQTVGVGTITGRGRGRLGTPRGRGGWTPLDRENMPPPGSARRRRGRGSN
SLLPSWYPRTPLRDITAVVRAIERRGRLGGSDGREIGSPMPQGRMDPEFS
EATPVAHPEPSNRIMSPKPTPAFKGCPSTIGKVPKILQHITNQASGDPEC
LTPQKKLLNSIDTVEKVVMEELQKLKRTPSAKKAEREKRVRTLMSMR
Populus Pt ABK93885 XP_002330993 (SEQ ID No. 30):
MPVSRDRLSSPVDIAALFAARRQSRILGVYQDQPELDMALFGSPRPNAAT
RTQTVGAGTIAVRGRGGLGTPRGRGGRTTLGRENIPPPGSARRGRGRGSN
SVLPAWYPRTPLRDVTAVVRAIERRRERLGGSDGLEIRSPMPQVRMNHDS
SEATPVAHLEHSNRIMSPKPTTAVKGCSSTIGKVPKILQHITNQASGDPD
SLTPQKKLLNSIDTVEKVVMEELRKMKRTPSARKAEREKRVRTLMSMR
Vitis Vv CAO23523 gi|225441692|ref|XP_002277253
(SEQ ID No. 31):
MPESRDRLSRPEDIAELFLRRRSGILGILADGSERSSNLFASPSRRETTT
RTTTLGARGATGILASRGGGVGRGGFGTPRIGTGRGRGRAVYRSPLFGRE
NTPATGSGRRGRGRSGNSVLPSWYPRTPLRDITHVVRAIERRRARLREID
GQQIDIPIPQDISDVHDPILPPSSAQLEQDISMISPSPTSGMKLVPKAVG
KVPKILLDITDQTGGGSDFLTPQKKLLNSIDTVEKAVMDELGKLKRTPSA
KRAEQEKRVRTLMSMR
Glycine max Gm|JGI_Gm0077x00122 (SEQ ID No. 32):
MPQSRHRRVTVVDLAASLARRRVSFIFNEAPTLRTPPRTAAFGRGRARAS
PRSQNIPPSTARRGRGRVPLRSVLPAWFPRTPLRDITAVVQAIERRSARL
GEVEGQRIGNTDPASDRLVSEPSEPASASASASAVKSPKSVGVKLRTPFG
SKVPKIFLDISELPEHDESEALTPQKKLLDNIDQVEEAVREELNKLKRTP
SAKKTEREKR
Glycine max Gm|JGI_Gm0128x00128 (SEQ ID No. 33):
MPESRDRRITVVDLAAAIARRRASFIYIDSPPLRTPQRTAAIGRGRASGS
PGSQNTPPSTARRGRGRVPSRNVLPAWYPRTPLRDITVVVQAIERRRARS
GEAEGQRIGSTDPASDRLVTEPSEPASADSAVKSPKSVGVKLRTPFGSKV
PKIFLDISELPEDDESETLTPQKKLLNNIDQVEEAVREELKKLKRTPSAK
KAEREKRVRTLMSMR
Oriza Os|CAH67433 Os04g39670 (SEQ ID No. 34):
MPEMRDSKRTALGELSGGGGFFIRRVASPGALAARGPGKPLARRFIR
PSNNKENVPPVWAVKATATKRRSPLPDWYPRTPLRDITAIAKAIQRSRLR
IAAAQQRSQTPEQNTPHCTEVRDSLDVEPGINSTQIVATPASSLAKDSLK
IFSSPSETSLVTPSKPMDPVLLDDMEKKLSSSIEQIEKMVRRNLKRTPKA
AAAQPSKRAIQRRTLMSMR
Sorghum Sb|JGI5057365 (SEQ ID No. 35):
MPDSRDGRRAALADLSSGVGGGGFFIRRVASPRALAVRGAGKPLARR
YMSPSRNKENLLPIWALRATPAKRSPLPGWYPRTPLRDITAIAKAIQRSR
ARIAAAQQQSQRIEQSPQSVNVTTPAQAEQDAPHIAEASHAVASGSGSTE
RETVANPATVLADDNLNVSSSPAESSLNTPSKPMDPALADIVEKKLSSSI
EKIEKLVRKNMKRTPKAARASRRATQRRNLMSMR
Sorghum Sb|JGI4979131 (SEQ ID No. 36):
MPQLRTASRPVLARNSTGGIFIRRRVASPGGAVKPLARRVRTHFSNK
ENVPPVGAARAKPKRRSPLPDWYPRSPLRDITSIVKALEKRNRLEEDAAR
QHIQWNEDSPQPVDPTTTVHAEHSDPDSQSTQTQETLGVVASPGSTSAVA
NNVTSVAEDKQEASSSPSDCLQMAPSKPNDPSPADLEKKMSSSIEQIEKM
VRRHMKETPKAAQPSKLVVQRRILMSMR
Sorghum Sb|JGI5055355 (SEQ ID No. 37):
MHESRTARRPALADISGGGFFIRRVESPGAVLVKGAVKPLARRALSQSSN
KENVPPVGAVRGAPKRKSPLPDWYPRTPLRDITSIVKAIERRSRLQNAAT
EQTILWTEDSSQSVDPITPASAEQGVPTIEGGQAVARHATSLGDGKLKTS
SSPFDCSLQATPSKPNDPALADLMEKKLSNSIEQIEKMVRRNLKKTPKAA
QPSKRTIQSRILMSMR
Zea mays Zm|ESTs (SEQ ID No. 38):
MPESRDGRSEDLADLSGGVGGGGFFIRRVASPGALAVRGVRKPLARRYIS
PSRNKENLLPVWALRVTPTKRSPLPGWYPRTPLRDITAIAKAIQRSRSRI
AAAQQRSQRIEQSSQSVNVTTPAQAEQDAHIAEASHAVASGSGSTEREAV
ANPATVLADDNLNVSSLAAEGSLNTPSKPMDPALADKKLSGSIEKVEKLV
RKNLKRTSRAAQASRRATQRRNLMSMR
Zea mays Zm|ESTs2 (SEQ ID No. 39):
MPQLRTASRPALASNSAGGFFIRRRVASPGTSQAKGAAKPLARRVRTPAA
RAKPKRRSPLPDWYPRVPLRDITSIVKALEKRNRLEEDAARQHIQSNEDS
SQPVDPTTAEHSDPDSQSTQTQETPGAVASGPSSTSAVANRVTSVAEGKQ
EATDCSLQVAPSKPNDPSPADLEKKLSGSIEQIEKMVRRHMKETHPKAAQ
PSKVVVQRRILMSMR
Zea mays Zm|ESTs3 (SEQ ID No. 40):
MLEVRTARRPALADISGGGFFMRTVESPGAVLVNGAVKRPARQFLSPSSN
KENVPPVGAFRATPKRRTPLPDWYPRTPLRDITSIVKAIERRRSRLQNAA
AQQQIQWTEDPSRSVDPITPVQAEQGGVPTTVDGQGVGSPATCLEDGKLK
TSSYPSSDCSLQATPSKPNDPALADLVEKRLSSSIEQIEKMVRRT
Medicago Mt|AC141114_13.2 (SEQ ID No. 41):
MPEARDRRVIPLDVDTLFRRPFSAVFQESEPLSVTPAPAPFTAGLDLFFT
ERTPVRREVARARRSPGSENTPPTTARRGRGRATASRSALPSWYPRTPLQ
DITAIVRAIERRRERQGTEEIEQTGTPVHANQLTIFSDPSSFSAAIGSSS
RVHKKSPKSCIKLKTPYGSKVPKIIIDIAKLPAAEDGESELLTPQKKLLH
SIDIIEREVKQELMKLKRTPTAKKAEHQKRVRTLMSMR
Mallus md|ESTs (SEQ ID No. 42):
MPEARDRLSRPVDLATAYAQRLAGNRRVYIDLPEQTILAFSPPVRLPTGL
GIGATGVVGVGGLPRSSLRTPRTVTGRGRISFRLSTVDRENTPSGSSHRR
RGRSSNSVLPSWYPRTPLHDITAVTRAIERRRARLAESNGENTEGQAPQD
QNALDQSLPVLGAQFDHGVPVTPYSALRTKRRLPPPVVKVQKIIRDVSNQ
PSEGEFLTPQKKLMNSIDMVEEVVRKELDRLKRTPSAK
Mallus md|ESTs2 (SEQ ID No. 43):
GRLPRSILRTPRTVTGRGRIPFRLSTVDRENTPRGSSHQRGGRASNSVLP
YWYPRSPLQDITAVVRAIESRRARLIESDGQNTEGQVPQDQNALDQSLPV
SGAQFDHGVPMTPYSAVRTKHCLPPSVGKVQQILRDVSNQPSEGEFLTPQ
KKLMNSIDMVEKVVTKELERLKRTPSSKKAEREQKVRTLMSMR
Ricinus communis gi|255583278|ref|XP_002532403.1
(SEQ ID No. 44):
MPEARDRLSRPIDIATVFSRRRSGLIGVYQDQPDLETALFGSPITSRLDT
ATRTGTVGLSPRGRGRGSFGTPRNQTLRGRHPYVTIGRENTPVTGRRGNG
NRSVLPSWYPRTPLRDITAIVRAIERRRELLGEGRAQEIESPVPHAYEVP
SDSSEPAVAHLEHSNSMMSPIPSLQVKRCPPTVGKVSKILLDITNKASDD
SEFLTPQKKLLNSIDTVEKEVMEELRKLKRTASAKKAEREKKVRTLMSLR
Tomato (Lycopersicon esculentum) (SEQ ID No. 45):
MAEGRDRLSRQEDPIDIYSRRRSMGRGGIEIFEDESPESSSRAPIQTAEA
RMAGTSGGRGGIGRIGFGSPRNRRGRNLFRTPARVIRQNISTQGRNRGRH
SVLPAWYPRTPRDITSIVRAERTRARLRESEGEQLESVVPQDHTDLGPSE
STSGAQLEHTNSLITPRPKTRSRYHTRSVGKVPKILLDITNQSTSEDAEC
LTPQRKLLNSIDTVEKHVMEELHKLKRTPSARKQERDKRVKTLMSMR
Melon MU51554 (SEQ ID No. 46):
MSEARDRLERQVDYAEVFARRRSEGILDEQEMGSNLIGTPIARATTTTAA
QQRPTNPGPGGGGANLRRTFGSPISGGIGRNRFLYRTPVLSRENPSAGSS
RRSRSRGRNSVLPIWYPRTPLRDITAVVRAIERTRARLRENEGQGSDSSP
SDAPERALEYSVSVASDHQEPIISLLTPKPTVGKVPKILRGIANENTVGA
ETLTPQKKLLNSIDKVEKVVMEELQKLKRTPSAKKAEREKRVRTLMSFR

TABLE 14
Oryza sativa Japonica Group OsPRD1 (NCBI
Accession No. CAE02100) (SEQ ID No. 47):

MSVQLHCLGI LLNSTKDAAT YIGDKQSLYL NLVNNLRLPR
LIPLHIDTFL ALRITLSDSI INLFWYSDEI RGEILFVLYK
LSLLNATPWD DICDNDNVDL SAIGRSLLQF SLEVLLKTQN
DDVRLNCIAL LLTLAKKGAF DILLLSDPSL INSAEAEDNV
PLNDSLVILF AEAVKGSLLS TNIEVQTGTL ELIFHFLSSD
ANIFVLKTLI DQNVADYVFE VLRLSGNNDP LVISSIKVLS
ILANSEERFK EKLAIAVSTL LPVLHYVSEI PFHPVQSQVL
RLVCISIINC SGILSLSQEE QIACTLSAIL RRHGNGELGM
SSETFALVCS MLVEILKLPS ADDIQKLPSF IVEASKHAIS
LTFSHEYDCL FLIPHSLLLL KEALIFCLEG NKDQILRKKS
LEDSIIETCE TYLLPWLESA IVDGNDEETL SGILQIFQII
LSRASDNKSF KFAEMLASSS WFSLSFGFMG LFPTDHVKSA
VYLVISSIVD KVLGISYGET IRDACIYLPP DPAELLYLLG
QCSSEDFNLA SCQCAILVIL YVCSFYNERL AADNQILASV
EQYILLNGAK FPHEIPGSLM LTLLVHLYAF VRGISFRFGI
PHSPEAEKTL FHAMTHKEWD LLLIRVHLIA LKWLFQNEEL
MEPLSFHLLN FCKFFCEDRT VMLSSSTQLV DIQLIAELVY
SGETCISSLL VSLLSQMIKE SAEDEVLSVV NVITEILVSF
PCTSDQFVSC GIVDALGSIY LSLCSSRIKS VCSLLIFNIL
HSASAMTFTC DDDAWLALTM KLLDCFNSSL AYTSSEQEWK
ILIGILCLIL NHSANKVLIE PAKAIILNNC LALLMDGIVQ
EACAKGPSLF QHNQETTFGE LLILMLLLIF FSVRSLQAIL
EASIDWQEFL QYSDDTESSS VLGIPCHDLC RLMHFGPSPV
KLIASQCLLE LLNRISDQRS CLNAELRCSA KYLKSMIAVT
EGMVFDQDSR VAENCGACLT VILGWERFGS REKAVIRESK
WSRLILEEFA VALTAPGLTS KSFSNQQKIA ANIALSLLQL
SQVPDWLTSL FSDSLISGIV ANLSARNVTA EIVTLFSELM
AKNYLNQEHI AGLHNLFQVC RRQAYEGGGG SKAQPSEQKA
AAARCADDVR ALLFGMMLEQ RACSRATVEM EQQRLLREID
SFFFQESSLR EQNSVK

TABLE 15
Oryra sativa Protein Sequences:

Oryza sativa SPO11-1 protein sequence GenBank AAP68363
(SEQ ID No. 48):
MAGREKRRRV AALDGEERRR RQEEAATLLH RIRGLVRWVV AEVAAGRSPT
VALHRYQNYC SSASAAAASP CACSYDVPVG TDVLSLLHRG SHASRLNVLL
RVLLVVQQLL QQNKHCSKRD IYYMYPSIFQ EQAVVDRAIN DICVLFKCSR
HNLNVVPVAK GLVMGWIRFL EGEKEVYCVT NVNAAFSIPV SIEAIKDVVS
VADYILIVEK ETVFQRLAND KFCERNRCIV ITGRGYPDIP TRRFLRYLVE
QLHLPVYCLV DADPYGFDIL ATYKFGSLQL AYDANFLRVP DIRWLGVFTS
DFEDYRLPDC CLLHLSSEDR RKAEGILSRC YLHREAPQWR LELEAMLQKG
VKFEIEALSA CSISFLSEEY IPKKIKQGRH I
Oryza sativa SPO11-2 protein sequence GenBank NP_001061027
(SEQ ID No. 49):
MAEAGVAAAS LFGADRRLCS ADILPPAEVR ARIEVAVLNF LAALTDPAAP
AISALPLISR GAANRGLRRA LLRDDVSSVY LSYASCKRSL TRANDAKAFV
RVWKVMEMCY KILGEGKLVT LRELFYTLLS ESPTYFTCQR HVNQTVQDVV
SLLRCTRQSL GIMASSRGAL IGRLVVQGPE EEHVDCSILG PSGHAITGDL
NVLSKLIFSS DARYIIVVEK DAIFQRLAED RIYSHLPCIL ITAKGYPDLA
TRFILHRLSQ TYPNMPIFAL VDWNPAGLAI LCTYKYGSIS MGLESYRYAC
NVKWLGLRGD DLQLIPQSAY QELKPRDLQI AKSLLSSKFL QDKHRAELTL
MLETGKRAEIEALYSHGFDF LGKYVARKIV QGDYI
Oryza sativa PAIR1 protein SwissProt Q75RY2 (SEQ ID NO. 50):
MKLKMNKACD IASISVLPPR RTGGSSGASA SGSVAVAVAS QPRSQPLSQS
QQSFSQGASA SLLHSQSQFS QVSLDDNLLT LLPSPTRDQR FGLHDDSSKR
MSSLPASSAS CAREESQLQL AKLPSNPVHR WNPSIADTRS GQVTNEDVER
KFQHLASSVH KMGMVVDSVQ SDVMQLNRAM KEASLDSGSI RQKIAVLESS
LQQILKGQDD LKALFGSSTK HNPDQTSVLN SLGSKLNEIS STLATLQTQM
QARQLQGDQT TVLNSNASKS NEISSTLATL QTQMQADIRQ LRCDVFRVFT
KEMEGVVRAI RSVNSRPAAM QMMADQSYQV PVSNGWTQIN QTPVAAGRSP
MNRAPVAAGR SRMNQLPETK VLSAHLVYPA KVTDLKPKVE QGKVKAAPQK
PFASSYYRVA PKQEEVAIRK VNIQVPAKKA PVSIIIESDD DSEGRASCVI
LKTETGSKEW KVTKQGTEEG LEILRRARKR RRREMQSIVL AS
Oryza sativa REC8 Gen bank AAQ75095 (SEQ ID No. 51):
MFYSHQLLAR KAPLGQIWMA ATLHSKINRK RLDKLDIIKI CEEILNPSVP
MALRLSGILM GGVAIVYERK VKALYDDVSR FLIEINEAWR VKPVADPTVL
PKGKTQAKYE AVTLPENIMD MDVEQPMLFS EADTTRFRGM RLEDLDDQYI
NVNLDDDDFS RAENHHQADA ENITLADNFG SGLGETDVFN RFERFDITDD
DATFNVTPDG HPQVPSNLVP SPPRQEDSPQ QQENHHAASS PLHEEAQQGG
ASVKNEQEQQ KMKGQQPAKS SKRKKRRKDD EVMMDNDQIM IPGNVYQTWL
KDPSSLITKR HRINSKVNLI RSIKIRDLMD LPLVSLISSL EKSPLEFYYP
KELMQLWKEC TEVKSPKAPS SGGQQSSSPE QQQRNLPPQA FPTQPQVDND
REMGFHPVDF ADDIEKLRGN TSGEYGRDYD AFHSDHSVTP GSPGLSRRSA
SSSGGSGRGF TQLDPEVQLP SGRSKRQHSS GKSFGNLDPV EEEFPFEQEL
RDFKMRRLSD VGPTPDLLEE IEPTQTPYEK KSNPIDQVTQ SIHSYLKLHF
DTPGASQSES LSQLAHGMTT AKAARLFYQA CVLATHDFIK VNQLEPYGDI
LISRGPKM

### The Examples

### Improved Ploidy Reducer

The GFP-tailswap plant (cenh3-1 mutant plants rescued by a GFP-tailswap transgene) is a very efficient haploid inducer, but is difficult to cross as the pollen donor, because it is mostly male sterile. Further, GFP-tailswap plants give an extremely low frequency of viable seeds (2%) when crossed as female to a tetraploid male that produces diploid gametes. In comparison, GFP-CENH3 (cenh3-1 mutant plants rescued by a GFP-tailswap transgene) is a weaker haploid inducer, but is much more fertile than GFP-tailswap (Ravi and Chan 2010).

In order to develop an efficient genome elimination strain with improved fertility and seed viability, cenh3-1 plants expressing combinations of CENH3 variants were screened. A cenh3-1 line that co-expresses two altered versions of the CENH3 protein, specifically GFP-CENH3 and GFP-tailswap, was found to produce more viable pollen and give better seed set than GFP-tailswap, yet still induces genome elimination when crossed to wild-type tetraploid plants and induced genome elimination in either direction of a cross. GEM is produced by crossing a GFP-tailswap plant with a GFP-CENH3 plant and selecting progeny which express both altered CENH3 proteins.

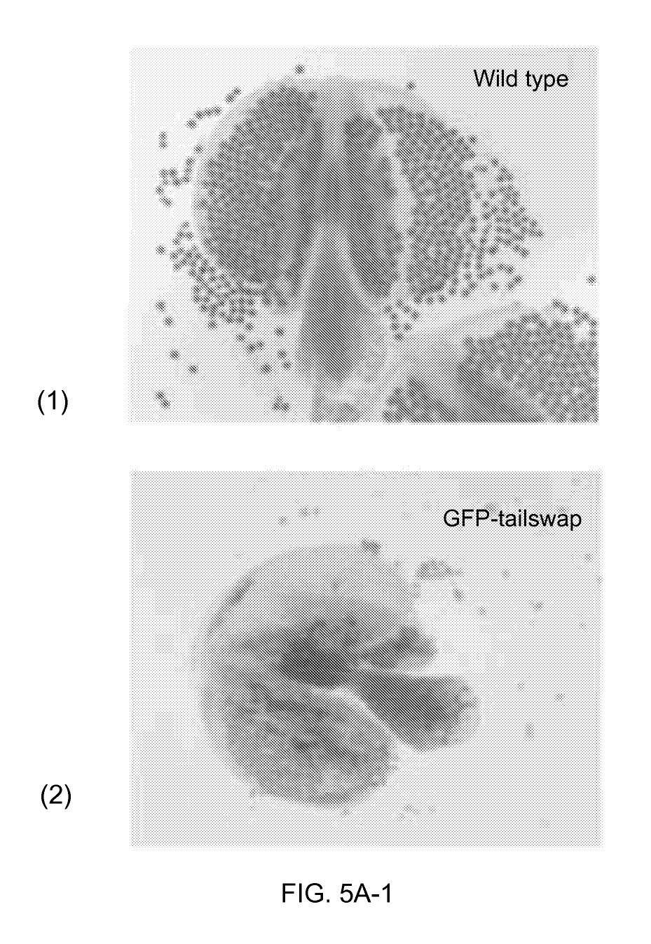

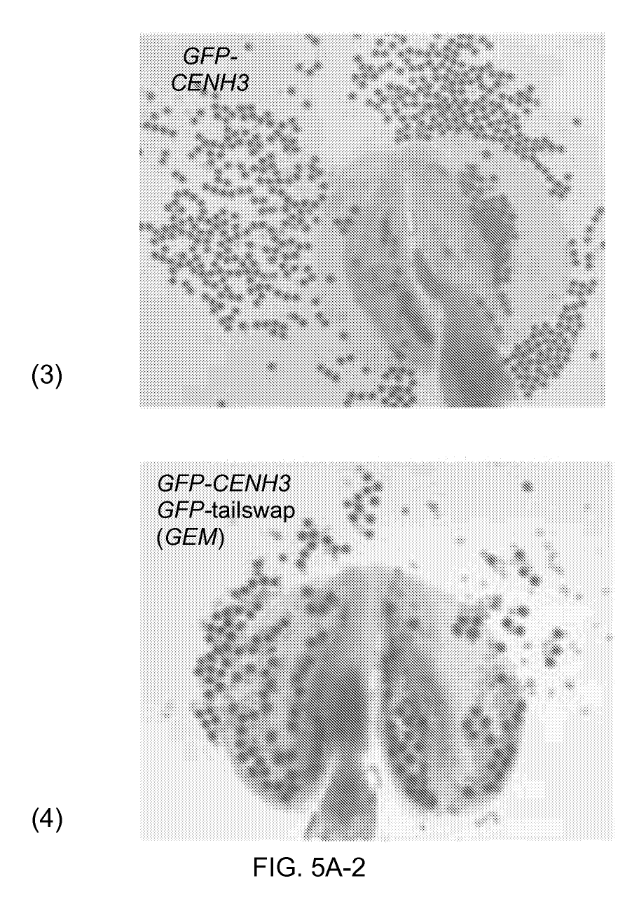

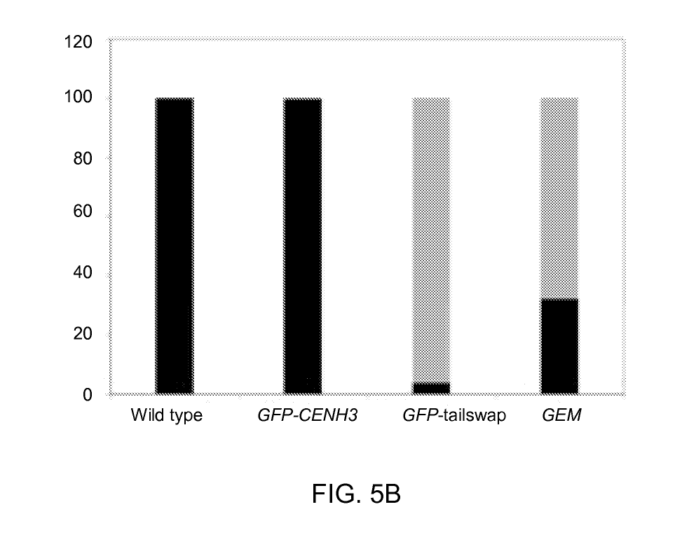

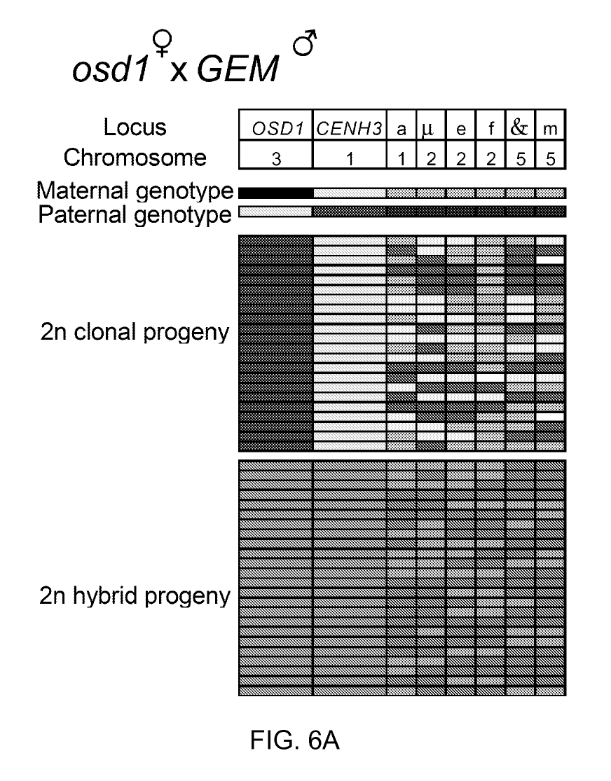

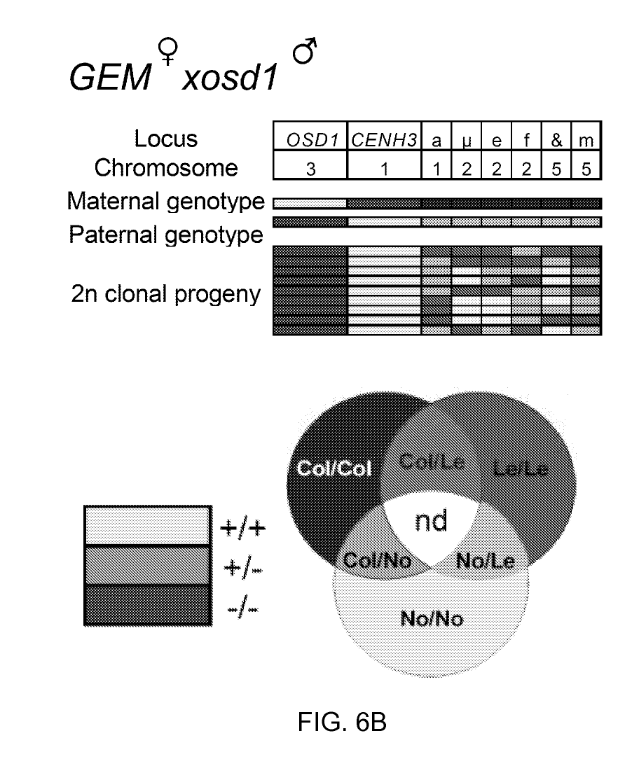

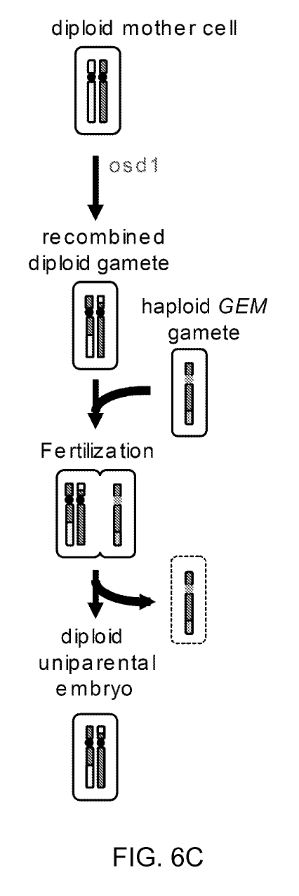

Indeed, cenh3-1 plants carrying both GFP-CENH3 and GFP-tailswap transgenes (GEM; Genome Elimination caused by a Mix of cenh3 variants) produced ample pollen for crosses, although pollen viability was still lower than wild-type (FIGS. 5 A and B) as shown by vital staining of pollen grains by Alexander staining (FIG. 5A). The graph of FIG. 5B shows the percentage of viable (black) and dead (grey) pollen from the genotyped indicated. When these co-expressing GEM plants were crossed as female or male to tetraploid wild-type, their chromosomes were eliminated in a subset of F1 progeny as shown in Table 16, see also FIGS. 6A-C. Further seed viability was much higher (40% and 80% higher, respectively) compared to the GFP-tailswap cross. In summary, GEM is fertile as either male or female, and shows efficient genome elimination when crossed to a parent with diploid gametes.

Detailed description of plants expressing certain altered CENH3 proteins are provided in Ravi, M. & Chan, S. W-L. (2010) and Ravi, M. et al. (Jul. 13, 2010), each of which is incorporated by reference herein in its entirety for such description. In particular these references provide detail description of the null mutant cenh3-1, GFP-tagged variants of CENH3, of GFP-CENH3, GFP-tailswap (in which endogenous CENH3 is replaced with a variant CENH3 in which the N-terminal tail domain of CENH3 is replaced with the N-terminal tail domain of H3 (centromere-specific histone H3). Heterologous CENH3 variants were expressed from the CENH3 promoter in some cases with an N-terminal GFP tagged.

Crosses Between osd1 and GEM Lead to Diploid Uniparental, but Recombined Progeny

Diploid mutants of osd1 produce diploid male and female gametes because of an absence of second division of meiosis (d'Erfurth, Jolivet et al. 2009). We have found that crossing osd1 to GEM gave rise to diploid progeny originated only from the diploid osd1 parent because of elimination of the GEM parent genome. This was demonstrated by taking advantage of the three different genetic backgrounds of the osd1-1 (No-0) and osd1-2 mutants (Ler) and GEM (Col-0). We crossed osd1-1/osd1-2 plants that were heterozygous for polymorphism between No-0 and Ler, to GEM and followed parental origin in the progeny using trimorphic markers.

Among the progeny issued from crosses between osd1 and GEM 13% were parthenogenetic and 20% were androgenetic, depending on the direction of the cross.

Crossing osd1-1/osd1-2 as female with GEM as male resulted in 29 viable seeds per fruit, 26% of them being diploid (Table 16). Among these diploid progeny, half (24/50) were from sexual origin, carrying alleles of both parents (FIG. 6A). These plants likely originate form the ˜20% of haploid female gametes produced by osd1 mutants (d'Erfurth, Jolivet et al. 2009). The other half of the diploid progeny (26/50) carried only maternal alleles at every locus tested (FIG. 6A). These diploid eliminant plants also exhibited the osd1 phenotype like their mother, having wild type somatic development and producing a dyad of spores instead of tetrad after meiosis. Moreover, the genotype of these plants perfectly reflected the genotype of the osd1-1/osd1-2 gametes. Indeed, because osd1 mutant gametes are produced following a single first division of meiosis, heterozygosity at centromeres is lost in the diploid gametes because of co-segregation of sister chromatid centromeres during this division. Because of recombination that occurs during the first division, any loci which are not linked to a centromere segregates in the osd1 diploid gametes (d'Erfurth, Jolivet et al. 2009). The genotypes of the diploid eliminant plants we obtained showed exactly this pattern (FIG. 6A, μ is a centromeric locus), confirming that their genome originated exclusively from osd1 diploid maternal gametes and that the plants are thus parthenogenic.

The possibility of androgenesis was tested by crossing GEM as female with osd1-1/osd1-2 as male. This resulted in 3-4 viable seeds per fruit (Table X), 20% of them being diploid suggestive of androgenesis, because osd1 produces only 2n pollen grains (d'Erfurth, Jolivet et al. 2009). All of these 2n plants carried exclusively paternal alleles (FIG. 6B) and exhibited the osd1 phenotype like their father. These diploid plants were thus from paternal origin. As in the previous cross, their genotype reflected the genotype of ods1 gametes, being recombined and having lost paternal heterozygosity in the vicinity of centromeres (FIG. 6B). These progeny are thus androgenetic having used GEM as a surrogate mother.

TABLE 16
Analysis of crosses between GEM and 4n Wild-type or osd1

| Cross | Seeds/ | Germination | Total Plants | Hybrid¹ | Triploid | Aneuploid | Uniparental |
|---|---|---|---|---|---|---|---|
| (female × male) | siliqua | Rate (%) | analyzed | Diploid (%) | (%) | (%) | diploid plants |
| Wild-type 4n × GEM | 35 | 81 | 85 | N/A | 62 | 32 | 6 |
| GEM × Wild-type 4n | 20 | 40 | 84 | N/A | 14 | 68 | 18 |
| osd1 × GEM | 31 | 93 | 196 | 26 | 31 | 43 | 13 |
| GEM × osd1 | 14 | 25 | 49 | 20 | 24 | 55 | 20 |

¹Deduced from FIGS. 6A-C. Tetraploid wild-type was in the C24 accession.

Crosses Between MiMe and GEM Lead to Diploid Uniparental Progeny

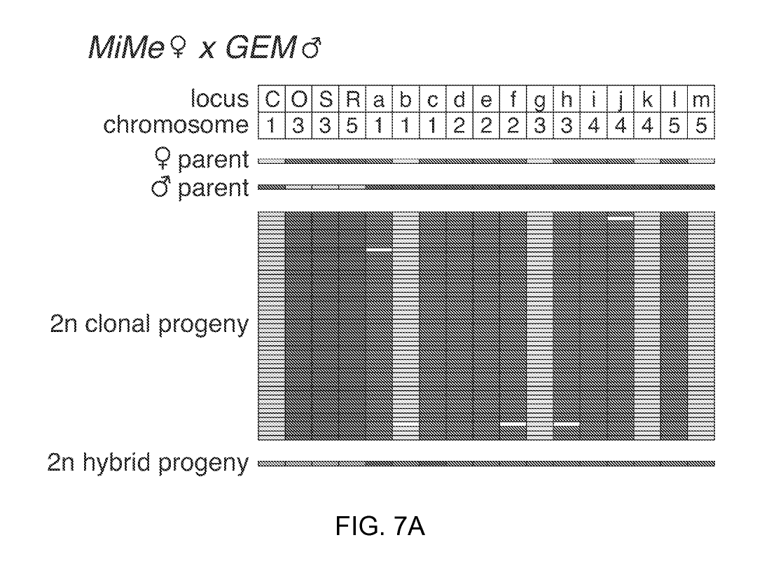

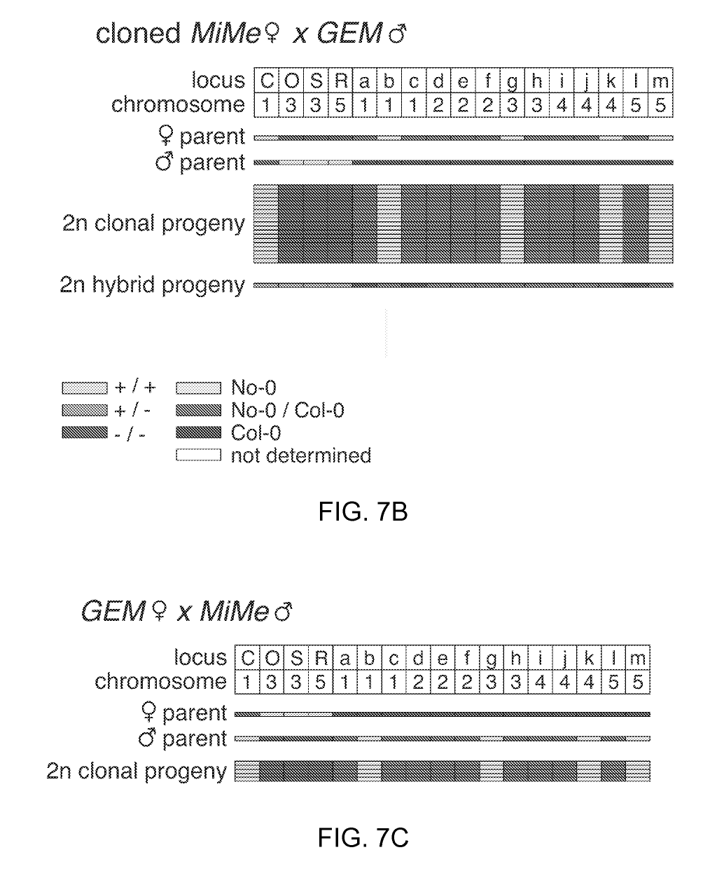

In this example we test the combination of apomeiosis with uniparental genome elimination. We crossed MiMe plants as female to the GEM line and looked for genome elimination events in the progeny. The MiMe parent had been previously genotyped and found to be either heterozygous or homozygous for a set of microsatellite markers across the genome (FIGS. 7A-C and Table 17). As the MiMe plants were from a mixed No-0 and Col-0 background, and GEM was pure Col-0 we could trace the origin of the chromosomes in the F1 progeny.

TABLE 17
List of markers used in this Example

| | | | |
|---|---|---|---|
| a | f5iI4 | n | NGA63 |
| b | msat1.13 | o | NGA280 |
| c | msat1.1 | p | NGA1145 |
| d | msat2.17 | q | NGA168 |
| e | msat2.21 | r | NGA 162 |
| f | msat2.9 | s | GAPAB |
| g | msat3.32 | t | NGA6 |
| h | msat3.07194 | u | NGA1107 |
| i | 4.02575 | v | NGA225 |
| j | 4.35 | w | CA72 |
| k | 4.18 | x | NGA139 |
| l | Ath5S0262 | y | SO262 |
| m | nga76 | z | CDC2A |
| μ | msat2.18 | & | NGA151 |
| α | NGA8 | | |

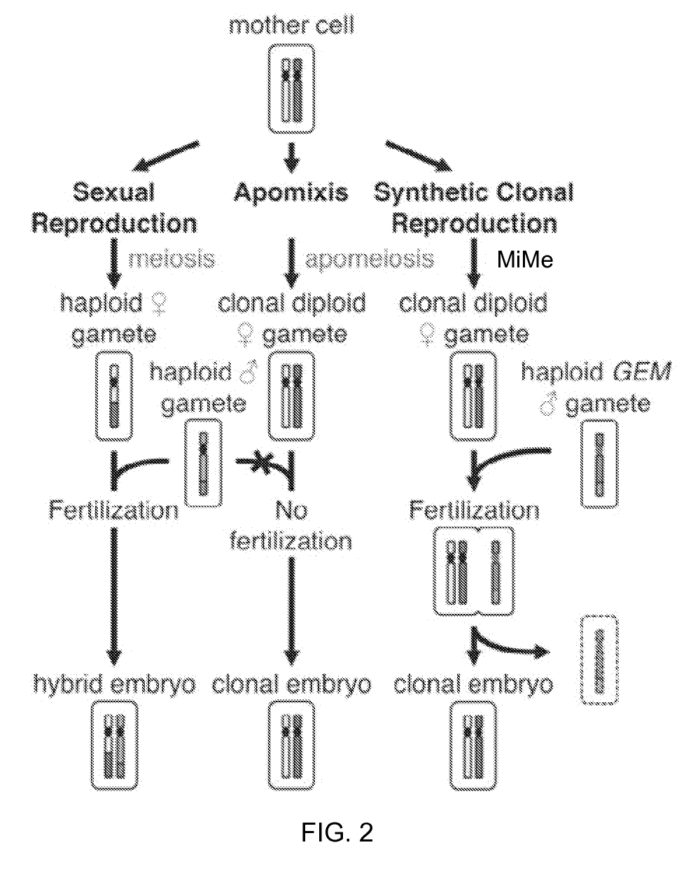

MiMe×GEM gave an average of 14 viable seeds per fruit (˜1/3 of wild type), 35% of them being diploid (Table 18). Among these 2n plants, 98% (51/52) were entirely of maternal origin, lacking paternal contribution for eight loci tested at which the parents were homozygous for distinct alleles (FIG. 7A). Diploid hybrid progeny in MiMe crosses probably result from haploid gametes fertilized by GEM sperm without genome elimination (FIGS. 7A and 7B). Furthermore, these diploid eliminants systematically kept the heterozygosity of the mother plant for all tested loci. For all crosses these results rule out post-elimination doubling following fertilization of a haploid gamete and show that genome elimination took place after fertilization of an unreduced female gamete that was apomeiotic, and that resulting plants were clones of the maternal parent (FIG. 7A). These results demonstrate engineering of clonal propagation through seed in a manner akin to the outcome of diplosporous or aposporous apomixis (FIG. 1 and FIG. 2).

MiMe also produces male apomeiotic gametes. We tested if MiMe plants could be cloned as male. The GEM line was crossed as a female to MiMe plants and the elimination events were characterized Although seed viability was much lower in this cross, likely due to the fact that the Col-0 strain is very sensitive to paternal genome excess [Dilkes, B. P. et al. (2008)], 42% of progeny were diploid (Table XII). They all lacked maternal contribution and systemically kept heterozygosity of the male parent for all tested loci (FIG. 7C). Thus these plants are clones of their MiMe father, having used GEM as a surrogate mother, mimicking the unique described case of male apomixis. [Pichot, C., et al. (2001)]

TABLE 18
Analysis of crosses between GEM and MiMe

| cross | Seeds per | Germination | Total plants | Hybrid | Triploid | Aneuploid | Clones * |
|---|---|---|---|---|---|---|---|
| ( [symbol] × [symbol] ) | siliqua | rate (%) | analysed | diploid¹ (%) | (%) | (%) | (%) |
| MiMe × GEM | 15 | 92 | 156 | 0.6 | 13 | 53 | 34 |
| GEM × MiMe | 23 | 0.5 | 12 | 0 | 25 | 33 | 42 |
| cloned MiMe × GEM | 14 | 91 | 79 | 1.3 | 20 | 54 | 24 |

¹Deduced from FIGS. 7A-C data.

Genotype Analysis of GEM×MiMe Progeny

FIG. 7A-C presents a summary of genotype analysis of GEM×MiMe progeny. Parents and diploid progeny were genotyped for parental mutations and polymorphic loci (Table 17). Each row represents one plant and each column is a locus. (A) MiMe [symbol] (female)×GEM [symbol] (male). Diploid plants were identified by flow cytometry, confirmed by mitotic chromosome spreads and genotyped. 51/52 had the same genotype as their mother (clonal progeny) and one had a hybrid genotype. (B) GEM [symbol] (female)×MiMe [symbol] (male). All diploid progeny had the same genotype as their mother. (E) Cloned MiMe [symbol] (female)×GEM [symbol] (male). One of the cloned plants shown in A was crossed to GEM [symbol] (male) and in the progeny 19/20 diploid plants had the same genotype as their mother and grandmother and one had a hybrid genotype.

Genotype Analysis of osd1×GEM and GEM×osd1 Offspring

As illustrated in FIGS. 6 A, B and C, diploid offspring of the crosses, identified by flow cytometry and confirmed by mitotic chromosome spreads, were genotyped for parental mutations and several trimorphic molecular markers (see Table 17). Each line (in FIGS. 6 A and B) represents one plant. For each mutation, the wild type genotype is represented in light grey, the heterozygote in medium grey, and the homozygote mutant genotype in dark grey. For each marker, the genotype is encoded according to the color rosace. Markers in white were not determined. For each cross, the two first lines represent the parental genotype. (A) osd1 [symbol] ×GEM [symbol] . Among the diploid plants, half had a genotype of maternal origin (recombined), lacking paternal contribution and the other half had a hybrid genotype. (B) GEM [symbol] ×osd1 [symbol] . Among the diploid plants, all had a genotype of paternal origin (recombined), lacking maternal contribution. FIG. 6C is a schematic representation of the mechanisms of production of diploid uniparental recombined progeny. Table 17 provides a list of markers used in this study.

Genotyping and Microsatellite Marker Analysis

Primers sequences and genotyping of plants for cenh3, GFP-tailswap, and GFP-CENH3 are listed below. Primers for osd1-1, Atspo11-1 and Atrec8-3 (MiMe) genotyping are described in [d'Erfurth, I. et al. (2009)]. Microsatellite markers (Table 17, above) were analyzed as described therein. [See also d'Erfurth, I. et al. (2008). and Dolezel, J et al. (2007)]. The cyclin-A CYCA1;2/TAM is required for the meiosis I to meiosis II transition and cooperates with OSD1 for the prophase to first meiotic division transition. Primer sequences were obtained from TAIR (www.arabidopsis.org) or from the MSAT database (INRA).

Identification of Diploid Plants from GEM×C24 Wild Type Tetraploid and its Reciprocal Cross

1. Putative diploid plants were first screened by their phenotype. Aneuploid plants can be morphologically distinguished from diploid and triploid plants. Triploid plants are hybrids containing Col-0 and C24 chromosomes. They are thus very late flowering, partially because of the combination of Col-0 FRIGIDA and C24 FLOWERING LOCUS C alleles [Sanda S. L. & Amasino R. M. (1995)]

2. All putative diploid plants along with randomly chosen sexual aneuploids and triploids were genotyped for at least one marker per chromosome. Pure diploids had only C24 alleles. Triploids had both C24 and Col-0 alleles. Aneuploids had all C24 alleles and lacked certainCol-0 alleles depending on the absence of a particular chromosome.

3. True diploid plants formed by genome elimination show a lack of GFP fluorescence because of the absence of GFP-tailswap whereas sexual aneuploids and triploids show GFP fluorescence at centromeres.

4. Random diploid plants were further confirmed by karyotyping in mitotic or meiotic spreads.

Diploid plants were genotyped to confirm their 4n C24 parental origin using the markers listed in Table 19

TABLE 19
Markers for Genotyping

| Chromosome No. | Marker |
|---|---|
| 1 | F5I14, CIW12 |
| 2 | MSAT2.1 |
| 3 | MSAT3.19, CIW11 |
| 4 | nga |
| 5 | CTR1.2, nga106 |

Genotyping the cenh3-1 Mutation and the GFP-Tailswap Transgene.

cenh3-1 is a point mutation in the CENH3 gene (also known as HTR12). The mutation is G161A relative to ATG=+1. cenh3-1 is genotyped with the following dCAPS primers:

Primer 1:
(SEQ ID No. 11)
GGTGCGATTTCTCCAGCAGTAAAAATC
Primer 2:
(SEQ ID No. 12)
CTGAGAAGATGAAGCACCGGCGATAT
(dCAPs restriction polymorphism with EcoRV)

GFP-tailswap is on chromosome 1 (identified by TAIL PCR). We genotype GFP-tailswap with the following primers:

Primer 3 for wild type and T-DNA:
(SEQ ID No. 13)
CACATACTCGCTACTGGTCAGAGAATC
Primer 4for wild type only:
(SEQ ID No. 14)
CTGAAGCTGAACCTTCGTCTCG
Primer 5 for the T-DNA:
(SEQ ID No. 15)
AATCCAGATCCCCCGAATTA

The presence of GFP-CENH3 can be detected using the following primers:

Primer 6:
(SEQ ID No. 16)
CAGCAGAACACCCCCATC (in GFP)
Primer 7:
(SEQ ID No. 17)
CTGAGAAGATGAAGCACCGGCGATAT (in CENH3)

Plant Material and Growth Conditions

Plants were grown in artificial soil mix at 20° C. under fluorescent lighting. Wild type and mutant strains of Arabidopsis were obtained from ABRC, Ohio or NASC, UK. MiMe plants were by construction a mixture of Col-0 from Atspo11-1/Atrec8 and No-0 from osd1-1 [d'Erfurth, I. et al. (2009)].

Ploidy Analysis

MiMe and osd1 offspring ploidy analyses were performed by flow cytometry and chromosome spreads as described [d'Erfurth, I. et al. (2009) and d'Erfurth, I. et al. (2010)].

### REFERENCES

Bains, G. S. & Howard, H. W. Haploid plants of Solanum demissum. Nature 166, 795 (1950).
Barclay, I. R. High frequencies of haploid production in wheat (Triticum aestivum) by chromosome elimination. Nature 256, 410-411 (1975).
Barret, P., Brinkmann, M., Beckert, M. A major locus expressed in the male gametophyte with incomplete penetrance is responsible for in situ gynogenesis in maize. Ther. Appl. Genet 117, 581-594 (2008).
Baulcombe, D. RNA silencing in plants Nature 431:356-363 (2004).
Bennett, M. D., Finch, R. A. & Barclay, I. R. The time rate and mechanism of chromosome elimination in Hordeum hybrids. Chromosoma, 54, 175-200 (1976).
Bicknell, R. A. & Koltunow, A. M. Understanding apomixis: recent advances and remaining conundrums. Plant Cell 16 Suppl, S228-45 (2004).
Bordes, J. R. et al., Haploidization of maize (Zea mays L.) through induced gynogenesis assisted by glossy markers and its use in breeding. Agronomie 17:291-297 (1997).
Burk, L. G., Gerstel, D. U. & Wernsman, E. A. Maternal haploids of Nicotiana tabacum L. from seed. Science 206, 585 (1979).
Chalyk, Bylich & Chebotar et al. MNL 68:47 (1994).
Chalyk & Chebotar Plant Breeding 119:363-364 (2000).
Chalyk, S. T. Properties of maternal haploid maize plants and potential application to maize breeding. Euphytica 79; 13-18 (1994).
Chelysheva L, Diallo S, & Vezon D, AtREC8 and AtSCC3 are essential to the monopolar orientation of the kinetochores during meiosis. Journal of Cell Science 118, 4621-4632. (2005).
Clausen, R. E. & Mann, M. C. Inheritance of Nicotiana tabacum. V. The occurrence of haploid plants in interspecific progenies. Proc. Natl Acad. Sci. USA 10, 121-124 (1924).
Coe E. H. A line of maize with high haploid frequency Am. Nat. 93:381-382 (1959).
Deimling S, Röber F K, Geiger H H Methodik and Genetik der in-vivo-Haploiden induktion bei Mais. Vortr. Pflanzenzüchtung 38:203-224 (1997).
De Muyt A, Pereira L, & Vezon D, et al. A high throughput genetic screen identifies new early meiotic recombination functions in Arabidopsis thaliana. PLoS Genetics 5, e1000654 (2009).
De Muyt A, Vezon D, Gendrot G, Gallois J L, Stevens R, Grelon M. AtPRD1 is required for meiotic double strand break formation in Arabidopsis thaliana. EMBO J. 26, 4126-4137 (2007).
d'Erfurth, I. et al. Mutations in AtPS1 (Arabidopsis thaliana parallel spindle 1) lead to the production of diploid pollen grains. PLoS Genet 4, e1000274 (2008).
d'Erfurth, I. et al. Turning meiosis into mitosis. PLoS Biol 7, e1000124 (2009).
d'Erfurth, I. et al. The cyclin-A CYCA1;2/TAM is required for the meiosis I to meiosis II transition and cooperates with OSD1 for the prophase to first meiotic division transition. PLoS Genet 6, e1000989 (2010).
Dilkes, B. P. et al. The maternally expressed WRKY transcription factor TTG2 controls lethality in interploidy crosses of Arabidopsis. PLoS Biol 6, 2707-20 (2008).
Dolezel, J., Greilhuber, J. & Suda, J. Estimation of nuclear DNA content in plants using flow cytometry. Nat Protoc 2, 2233-44 (2007).
Dunwell, J. M. Haploids in flowering plants: origins and exploitation. Plant Biotechnol J 8, 377-424 (2010).
Eder J. and S. Chalyk, 2002, In vivo haploid induction in maize. Theor. Appl. Genet. 104:703-708 (2002).
Finch, R. A. Tissue-specific elimination of alternative whole parental genomes in one barley hybrid. Chromosoma 88, 386-393 (1983).
Geiger H. H. & Gordillo, G. A. Doubled haploids in hybrid maize breeding Maydica 54: 485-499 (2009).
Grelon M, Vezon D, Gendrot G, & Pelletier G. AtSPO11-1 is necessary for efficient meiotic recombination in plants. EMBO Journal 20, 589-600 (2001).
Guitton, A. E. & Berger, F. Loss of function of multicopy suppressor of IRA 1 produces nonviable parthenogenetic embryos in Arabidopsis. Curr\Biol 15, 750-4 (2005).
Hartung F, Wurz-Wildersinn R, Fuchs J, Schubert I, Suer S, & Puchta H. The catalytically active tyrosine residues of both SPO11-1 and SPO11-2 are required for meiotic double-strand break induction in Arabidopsis. The Plant Cell 19, 3090-3099 (2007).
Hougas, H. W. & Peloquin, S. J. A haploid plant of the potato variety Katandin. Nature 180, 1209-1210 (1957).
Kasha, K. J. & Kao, K. N. High frequency haploid production in barley (Hordeum vulgare L.). Nature 225, 874-876 (1970).
Kermicle, J. L. Science 166; 1422-24 (1969).
Klimyuk V. I. & Jones J D. AtDMC1, the Arabidopsis homologue of the yeast DMC1 gene: Characterization, transposon-induced allelic variation and meiosis-associated expression. Plant J. January; 1 1 (1):1-14 (1997).
Koltunow, A. M. & Grossniklaus, U. Apomixis: a developmental perspective. Annu Rev Plant Biol 54, 547-74 (2003).
La, H., Li, J., Ji, Z., Cheng, Y., Li, X., Jiang, S., Venkatesh, P. N. & Ramachandran, S. Genome-wide analysis of cyclin family in rice (Oryza Sativa L.) Mol. Gen Genomics 275:374-386 (2006).
Lashermes, P. & Beckert, M. Genetic control of maternal haploidy in maize (Zea mays L.) and selection of haploid inducing lines Theor Appl Genet 76:405-410 (1988).
Laurie, D. A. & Bennett, M. D. The timing of chromosome elimination in hexaploid wheat×maize crosses. Genome 32, 953-961 (1989).
Magnard, J.-L., Yang, M., Chen, Y.-C. S., Leary, M. & McCormick, S. The Arabidopsis gene Tardy Asynchronous Meiosis is required for the normal pace and synchrony of cell division during male meiosis Plant Physiol. 127:1157-1166 (2001).
Marimuthu M. P., Jolivet S., Ravi M., Pereira L., Davda J. N., Cromer L., Wang L., Nogué F., Chan S. W., Siddiqi I., Mercier R. Synthetic clonal reproduction through seeds. Science. 2011 Feb. 18, 331(6019):876.
McCallum C. M., Comai, L., Greene, E. A., & Henikoff, S. Targeting Induced Local Lesions IN Genomes (TILLING) for Plant Functional Genomics Plant Physiol, Vol. 123, pp. 439-442 (2000).
Mercier, R. & Grelon M. Meiosis in plants: ten years of gene discovery Cytogeneti Genome Res 120:281-290 (2008).
Nonomura K, Nakano M, Fukuda T, Eiguchi M, & Miyao A, The novel gene Homologous Pairing Aberration In Rice Meiosis1 of rice encodes a putative coiled-coil protein required for homologous chromosome pairing in meiosis. Plant Cell 16: 1008-1020 (2004).
Ossowski et al., Plant J., 53, 674-90 (2008).
Ozias-Akins, P. & van Dijk, P. J. Mendelian genetics of apomixis in plants. Annu Rev Genet 41, 509-37 (2007).
Pichot, C., El Maataoui, M., Raddi, S. & Raddi, P. Surrogate mother for endangered Cupressus. Nature 412, 39 (2001).
Ravi, M., Kwong, P. N., Menorca, R. M. G., Valencia, J. T., Ramahi, J. S., Stewart, J. L., Tran, R. K., Sundaresan, V., Comai, L. & Chan, S. W.-L. The rapidly evolving centromere-specific histone has stringent functional requirements in Arabidopsis thaliana. Genetics 186:461-471 (2010) (published on-line Jul. 13, 2010).
Ravi, M. & Chan, S. W. Haploid plants produced by centromere-mediated genome elimination. Nature 464, 615-8 (2010).
Röber F. K., Gordillo, G. A. & Geiger H. H., In vivo haploid induction in maize—performance of new inducers and significance of doubled haploid lines in Hybrid Breeding Maydica 50:275-283 (2005).
Rodrigues, J. C., Luo, M., Berger, F. & Koltunow, A. M. Polycomb group gene function in sexual and asexual seed development in angiosperms. Sex Plant Reprod 23, 123-33 (2010).
Sanda S. L. & Amasino R. M. Genetic and physiological analysis of flowering time in the C24 line of Arabidopsis thaliana Weeds World Volume 2(iii) (1995).
Sarkar K. R. &Coe E. H. A genetic analysis of the origin of maternal haploids in maize Genetics 54:453-464 (1966).
Sarkar K. R. et al, 1972, Development of maternal-haploidy-inducer lines in maize (Zea mays L.) Indian J. Agric. Sci. 42:781-786 (1972).
Savidan, Y. in The Flowering of Apomixis: From Mechanisms to Genetic Engineering (eds. Savidan, Y., Carman, J. & Dresselhaus, T.) (CIMMYT, IRD, Eur. Comm. DG VI (FAIR), Mexico)(2001).
Spillane, C., Curtis, M. D. & Grossniklaus, U. Apomixis technology development-virgin births in farmers' fields? Nat Biotechnol 22, 687-91 (2004).
Spillane, C., Steimer, A. & Grossniklaus, U. Apomixis in agriculture: the quest for clonal seeds. Sexual Plant Reproduction 14 (2001).
Stacey N J, Kuromori T, Azumi Y, Roberts G, Breuer C, Wada T, Maxwell A, Roberts K, & Sugimoto-Shirasu K. Arabidopsis SPO11-2 functions with SPO11-1 in meiotic recombination. The Plant Journal, 48, 206-216 (2006).
Watson, J. M., Fusaro, A. F., Wang, M. B., & Waterhouse, P. M. RNA silencing platforms in plants. FEBS Lett. 579:5982-5987 (2005).

1. A method for production of clonal embryos or seeds by conversion of apomeiotic gametes of a MiMe (mitosis instead of meiosis) plant into clonal embryos or seeds by crossing the MiMe plant with a plant that induces genome elimination and selecting embryos or seeds of plants resulting from the crossing which are clones of the MiMe plants.

2. The method of claim 1 wherein the plant that induces genome elimination exhibits a rate of haploid induction of 1% or higher.

3. The method of claim 1 wherein the crossing is performed by pollinating the MiMe plant with pollen of the plant that induces genome elimination.

4. The method of claim 1 wherein the crossing is performed by pollinating the plant that induces genome elimination with pollen of the MiMe plant.

5. The method of claim 1 wherein the plant that induces genome elimination is a plant expressing one or more altered centromeric-specific histone variant CENH3 proteins.

6. The method of claim 1 wherein the plant that induces genome elimination is a plant expressing two or more altered CENH3 proteins

7. The method of claim 1 wherein the plant that induces genome elimination co-expresses a tagged-endogenous CENH3 protein and a tagged CENH3 protein in which the N-terminal region of the endogenous CENH3 protein is replaced with the N-terminal region of a centromere specific histone protein other than the endogenous CENH3.

8. The method of claim 1 wherein the plant that induces genome elimination co-expresses tagged-tailswap or tagged-CENH3.

9. The method of claim 1 wherein the plant that induces genome elimination co-expresses tagged-tailswap or tagged-CENH3 is a mutant plant.

10. The method of claim 1 wherein the plant that induces genome elimination co-expresses tagged-tailswap or tagged-CENH3 is a transformed plant.

11. The method of claim 7 wherein the tag is Green Florescent Protein (GFP).

12. The method of claim 1 wherein the plants are Arabidopsis or Oryza.

13. The method of claim 1 wherein the plants are Arabidopsis thaliana or Oryza sativa.

14. The method of claim 1 wherein the plants are rice, soybean, corn or maize, rye, cotton, oats, barley, wheat, alfalfa, sorghum, sunflower, various legumes, various Brassica, potato, peanuts, clover, sweet potato, cassava (manioc), rye-grass, banana, melon, watermelon, sugar beets, sugar cane, lettuce, carrots, spinach, endive, leeks, celery, artichokes, beets, radishes, turnips or tomato or ornamental plants such as roses, lilies, tulips or narcissus.

15. The method of claim 1 wherein the plants are maize.

16. The method of claim 15 wherein the plant that induces genome elimination is selected from one of the maize lines PK6, RWS, RWK-76, FIGH 1 or derivatives thereof which retain the haploid inducer phenotype.

17. A method of plant breeding employing clonal seeds obtained by the methods of claim 1.

18. A method for cultivating a clonal plant that comprises the steps of:
generating clonal seed by the method of claim 1, cultivating a clonal plant from the clonal seed and recovering viable gametes from the cultivated plant.

19. Clonal progeny and plant cells and tissue thereof produced by crossing a MiMe plant with a genome eliminator plant.

20. The clonal progeny of claim 19 wherein the plant that is a genome eliminator plant is a plant expressing one or more altered centromeric-specific histone variant CENH3 proteins.
